SEQUENCE LISTING (21 sequences)

SEQ ID NO: 1 - protein, 8 aa, synthetic construct
  REGION 1..8: note = CDR1 A11
GNIFSINA

SEQ ID NO: 2 - protein, 7 aa, synthetic construct
  REGION 1..7: note = CDR2 A11
ITSGGTT

SEQ ID NO: 3 - protein, 18 aa, synthetic construct
  REGION 1..18: note = CDR3 A11
NADGYYSDYDKNLAEFNS

SEQ ID NO: 4 - protein, 8 aa, synthetic construct
  REGION 1..8: note = CDR1 C5
GRRFMVVA

SEQ ID NO: 5 - protein, 7 aa, synthetic construct
  REGION 1..7: note = CDR2 C5
ITNGGTT

SEQ ID NO: 6 - protein, 16 aa, synthetic construct
  REGION 1..16: note = CDR3 C5
TTDVVFRDGNGQIQSN

SEQ ID NO: 7 - protein, 7 aa, synthetic construct
  REGION 1..7: note = Consensus CDR2
  VAR_SEQ 3: note = any natural amino acid
ITXGGTT

SEQ ID NO: 8 - protein, 7 aa, synthetic construct
  REGION 1..7: note = Consensus CDR2
  VAR_SEQ 3: note = X is Ser or Asp
ITXGGTT

SEQ ID NO: 9 - protein, 124 aa, synthetic construct
  REGION 1..124: note = A11 VHH
QVQLVETGGGLVQAGGSLRLSCAASGNIFSINAMDWYRQAPGKERELVAAITSGGTTNYADSVKGRFTISRDNAKDTVYLQMNSLKPEDAAFYYCNADGYYSDYDKNLAEFNSWGQGTQVTVSS

SEQ ID NO: 10 - protein, 122 aa, synthetic construct
  REGION 1..122: note = C5 VHH
QVQLVESGGGLVQAGGSLRLSCAASGRRFMVVAMGWYRQAPGNQRELVASITNGGTTNYAGSVKGRFTISGDSAKNTVYLHMNSLKPEDTAVYYCTTDVVFRDGNGQIQSNWGQGTQVTVSS

SEQ ID NO: 11 - protein, 137 aa, synthetic construct
  REGION 1..137: note = A11 including C-terminal His6-tag
QVQLVETGGGLVQAGGSLRLSCAASGNIFSINAMDWYRQAPGKERELVAAITSGGTTNYADSVKGRFTISRDNAKDTVYLQMNSLKPEDAAFYYCNADGYYSDYDKNLAEFNSWGQGTQVTVSSGGLPETGHHHHHH

SEQ ID NO: 12 - protein, 25 aa, synthetic construct
  REGION 1..25: note = A11 FR1
QVQLVETGGGLVQAGGSLRLSCAAS

SEQ ID NO: 13 - protein, 17 aa, synthetic construct
  REGION 1..17: note = A11 FR2
MDWYRQAPGKERELVAA

SEQ ID NO: 14 - protein, 38 aa, synthetic construct
  REGION 1..38: note = A11 FR3
NYADSVKGRFTISRDNAKDTVYLQMNSLKPEDAAFYYC

SEQ ID NO: 15 - protein, 11 aa, synthetic construct
  REGION 1..11: note = A11 FR4
WGQGTQVTVSS

SEQ ID NO: 16 - protein, 25 aa, synthetic construct
  REGION 1..25: note = C5 FR1
QVQLVESGGGLVQAGGSLRLSCAAS

SEQ ID NO: 17 - protein, 17 aa, synthetic construct
  REGION 1..17: note = C5 FR2
MGWYRQAPGNQRELVAS

SEQ ID NO: 18 - protein, 38 aa, synthetic construct
  REGION 1..38: note = C5 FR3
NYAGSVKGRFTISGDSAKNTVYLHMNSLKPEDTAVYYC

SEQ ID NO: 19 - protein, 11 aa, synthetic construct
  REGION 1..11: note = C5 FR4
WGQGTQVTVSS

SEQ ID NO: 20 - protein, 135 aa, synthetic construct
  REGION 1..135: note = C5 including C-terminal His6-tag
QVQLVESGGGLVQAGGSLRLSCAASGRRFMVVAMGWYRQAPGNQRELVASITNGGTTNYAGSVKGRFTISGDSAKNTVYLHMNSLKPEDTAVYYCTTDVVFRDGNGQIQSNWGQGTQVTVSSGGLPETGHHHHHH

SEQ ID NO: 21 - protein, 7 aa, synthetic construct
  REGION 1..7: note = linker sequence
GGLPETG

(19) United States
(12) Patent Application Publication
(10) Pub. No.: US 2025/0179215 A1
(43) Pub. Date: Jun. 5, 2025

(54) PLASMIN-BINDING VHHS

(71) Applicant: RHEINISCHE FRIEDRICH-WILHELMS-UNIVERSITÄT BONN, Bonn (DE)

(72) Inventors: Bernd Pötzsch, Bonn (DE); Nasim Shahidi Hamedani, Alfter (DE); Eva-Maria Klein, Remagen (DE); Jens Müller, Düsseldorf (DE); Paul-Albert König, Bonn (DE); Florian Ingo Schmidt, Bonn (DE); Jan Moritz Paul Tödtmann, Bonn (DE)

(21) Appl. No.: 18/934,982

(22) Filed: Nov. 1, 2024

Related U.S. Application Data
(63) Continuation of application No. PCT/EP2023/061651, filed on May 3, 2023.

(30) Foreign Application Priority Data
May 4, 2022 (EP) 22171643.4

(51) Int. Cl. C07K 16/40; A61K 45/06; A61P 7/02

(52) U.S. Cl. CPC C07K 16/40; A61K 45/06; A61P 7/02; C07K 2317/31; C07K 2317/565; C07K 2317/569; C07K 2317/92

(57) ABSTRACT

The invention relates to a VHH specifically binding to plasmin. The invention further relates to a compound or a pharmaceutical composition comprising the VHH and to the use thereof in the treatment, prevention or amelioration of a disease or condition characterized by a thrombotic or embolic state.

### CROSS REFERENCE TO RELATED APPLICATIONS

This application claims the benefit of International Patent Application No. PCT/EP2023/061651 filed on May 3, 2023, which claims the benefit of European Patent Application No. 22171643.4 filed on May 4, 2022.

### SUBMISSION OF SEQUENCE LISTING XML

The content of the following submission on Sequence Listing XML is incorporated herein by reference in its entirety: a computer readable form (CRF) of the Sequence Listing (file name: P74166PC_sequence listing WIPO St 26.XML, date created Jan. 8, 2025, size: 28,412 bytes).

The invention relates to a VHH-single domain antibody (VHH) specifically binding to plasmin, as well as to a VHH specifically binding to plasmin wherein the VHH is a thrombolytic and/or fibrinolysis-activating VHH. The invention further relates to a compound or a pharmaceutical composition comprising the VHH and to the use thereof in the treatment, prevention or amelioration of a disease or condition characterized by a thrombotic or embolic state.

The fibrinolytic system is involved in a variety of biological processes including intravascular thrombolysis, inflammation, cell migration, and tissue remodeling (Anglés-Cano 1994, Baker 2020). Plasmin, the key enzyme of the fibrinolytic system, is generated from its inactive proenzyme plasminogen through limited proteolysis catalysed by the tissue plasminogen activator (tPA) or the urokinase plasminogen activator (uPA). Plasmin is composed of a C-terminal light chain that contains the serine protease domain and a N-terminal heavy chain or A-chain that bears five homologous kringle domains (Ponting 1992). These kringle domains play a critical role as binding mediators for a variety of molecules including plasma proteins and cellular receptors (Castellino 1997, Cao 2002). Thus, kringle domain 5 mediates binding of Glu-plasminogen to fibrin and is involved in interaction of plasmin with endothelial cells (Cao 1997, Ji 1998). Most likely, kringle domain-binding induces an intramolecular change that transists plasminogen from a closed spiral form into a semi-opened form that is more readily cleaved by tPA (Tharp 2009).

Activation of plasmin from its inactive proenzyme plasminogen is a prerequisite to fibrinolysis which, in turn, mediates clot resolution after embolism and thrombotic stroke or improves the endogenous lytic capacity in microthrombotic disorders such as venous occlusive disease or microthrombotic ischemia.

Inducing or increasing the activity of the plasminogen-plasmin system as well as lowering the rate of plasmin inactivation would, therefore, be desirable in order to improve emergency lysis in patients with acute thrombotic or embolic state and risk factors for residual emboli.

It was therefore an object of the present invention to provide means for inducing and/or increasing the activity of the plasminogen-plasmin system and/or means for lowering the rate of plasmin inactivation.

According to the invention, it was surprisingly found that a VHH specifically binding to plasmin may modulate the fibrinolytic and/or thrombolytic activity. In particular, the VHH of the invention which specifically binds to plasmin accelerates and/or increases plasmin generation and increases fibrin degradation. Thus, the VHH of the invention provides a new class of thrombolytic agents.

Accordingly, in one aspect, the invention relates to a VHH specifically binding to plasmin comprising complementarity determining regions (CDRs) CDR1, CDR2 and CDR3, wherein:
(a) CDR1 comprises or consists of the amino acid sequence GNIFSINA (SEQ ID NO: 1) or GRRFMWA (SEQ ID NO: 4);
(b) CDR2 comprises or consists of the amino acid sequence ITXGGTT (SEQ ID NO: 7) wherein X is a natural amino acid; and
(c) CDR3 comprises or consists of the amino acid sequence

(SEQ ID NO: 3)
NADGYYSDYDKNLAEFNS
or
(SEQ ID NO: 6)
TTDVVFRDGNGQIQSN.

A VHH single-domain antibody (VHH), is an antibody fragment consisting of a single monomeric variable antibody domain.

Just as a whole antibody, a VHH is able to bind selectively to a specific antigen. With a molecular weight of only 12-15 kDa, VVHs are much smaller than common antibodies (150-160 kDa) which are composed of two heavy chains and two light chains, and even smaller than Fab fragments (about 50 kDa, one light chain and half a heavy chain) and single-chain variable fragments (about 25 kDa, two variable domains, one from a light and one from a heavy chain) (Harmsen M M, De Haard H J (November 2007). “Properties, production, and applications of camelid single-domain antibody fragments”. Applied Microbiology and Biotechnology. 77 (1): 13-22).

The first VHHs were engineered from heavy-chain antibodies found in camelids and were referred to as “V_{H}H fragments” or just “VHHs”. Cartilaginous fishes also have heavy-chain antibodies (IgNAR, “immunoglobulin new antigen receptor”), from which single-domain antibodies referred to as “VNAR fragments” can be obtained (English H, Hong J, Ho M (January 2020). “NAR single domain antibody sequences, phage libraries and potential clinical applications”. Antibody Therapeutics. 3 (1): 1-9). An alternative approach is to split the dimeric variable domains from common immunoglobulin G (IgG) from humans or mice into monomers. Although most research into single-domain antibodies is currently based on heavy chain variable domains, VHHs derived from light chains have also been shown to bind specifically to target epitopes (Möller A, Pion E, Narayan V, Ball K L (December 2010). “Intracellular activation of interferon regulatory factor-1 by nanobodies to the multifunctional (Mf1) domain”. The Journal of Biological Chemistry. 285 (49): 38348-61).

Camelid VHHs have been shown to be just as specific as antibodies, and in some cases they are more robust. They are easily isolated using the same phage panning procedure used for antibodies, allowing them to be cultured in vitro in large concentrations. The smaller size and single domain structure make these antibodies easier to transform into bacterial cells for bulk production, making them ideal for research purposes (Ghannam A, Kumari S, Muyldermans S, Abbady A Q (March 2015). “Camelid nanobodies with high affinity for broad bean mottle virus: a possible promising tool to immunomodulate plant resistance against viruses”. Plant Molecular Biology. 87 (4-5): 355-69).

Hence, VHHs provide a promising alternative to “classical” antibodies, i.e. common antibodies which are composed of two heavy protein chains and two light chains. Due to their comparatively low molecular mass, VHHs exhibit a better permeability into tissues and, therefore, can diffuse in various organs and cross the blood brain barrier.

According to the invention, VHHs were identified that bind with high affinity to plasmin and increase the fibrinolytic activity of the plasminogen-plasmin system in a non-competitive manner. These fibrinolysis-activating VHHs offer a wide-range of pharmaceutical applications including improvement of emergency lysis in patients presented with life threatening lung embolisms and thrombotic stroke or improvement of the endogenous lytic capacity in microthrombotic disorders such as venous occlusive disease or microthrombotic ischemia.

As used herein, the term “VHH” refers to a single-domain antibody (sdAb) comprising one variable domain (V_{H}) of a heavy-chain antibody, or of a common IgG. The VHHs are also referred to “nanobodies” in the art.

From a structural point of view, the variable region, i.e. the VHH, comprises or consists of seven amino acid regions, four of which are framework regions (“FRs”) and three of which are complementarity determining regions (“CDRs”), also referred to as hypervariable regions. The framework regions are responsible for acting as a scaffold for the CDRs. The CDRs are in direct contact with the antigen and are involved in antigen binding. From the amino to the carboxy terminus the variable region, i.e. the VHH, comprises the framework regions and the complementarity determining regions in the following order:
FR1-CDR1-FR2-CDR2-FR3-CDR3-FR4

The VHH of the present invention specifically binds to plasmin.

As used herein, the term “plasmin” refers to a serine protease present in blood that acts to dissolve fibrin blood clots. Apart from fibrinolysis, plasmin proteolyzes proteins in various other systems: It activates collagenases, some mediators of the complement system, and weakens the wall of the Graafian follicle, leading to ovulation. Plasmin is also integrally involved in inflammation. It cleaves fibrin, fibronectin, thrombospondin, laminin, and von Willebrand factor.

Plasmin is released as a zymogen called plasminogen (PLG) from the liver into the systemic circulation. In circulation, plasminogen adopts a closed, activation-resistant conformation. Upon binding to clots, or to the cell surface, plasminogen adopts an open form that can be converted into active plasmin by a variety of enzymes, including tissue plasminogen activator (tPA), urokinase plasminogen activator (uPA), kallikrein, and factor XII (Hageman factor). Fibrin is a cofactor for plasminogen activation by tPA. Urokinase plasminogen activator receptor (uPAR) is a cofactor for plasminogen activation by uPA. Plasmin is inactivated by proteins such as α2-macroglobulin and alpha2-antiplasmin (α2AP) (Wu, Guojie; Quek, Adam J.; Caradoc-Davies, Tom T.; Ekkel, Sue M.; Mazzitelli, Blake; Whisstock, James C.; Law, Ruby H. P. (2019 Mar. 5). “Structural studies of plasmin inhibition”. Biochemical Society Transactions. 47 (2): 541-557).

According to the invention plasmin is preferably human plasmin.

In one embodiment the VHH specifically binding to plasmin comprises complementarity determining regions (CDRs) CDR1, CDR2 and CDR3, wherein:
(a) CDR1 comprises or consists of the amino acid sequence GNIFSINA (SEQ ID NO: 1);
(b) CDR2 comprises or consists of the amino acid sequence ITXGGTT (SEQ ID NO: 7) wherein X is a natural amino acid; and
(c) CDR3 comprises or consists of the amino acid sequence

(SEQ ID NO: 3)
NADGYYSDYDKNLAEFNS.

In another embodiment the VHH specifically binding to plasmin comprises complementarity determining regions (CDRs) CDR1, CDR2 and CDR3, wherein:
(a) CDR1 comprises or consists of the amino acid sequence GRRFMVVA (SEQ ID NO: 4);
(b) CDR2 comprises or consists of the amino acid sequence ITXGGTT (SEQ ID NO: 7) wherein X is a natural amino acid; and
(c) CDR3 comprises or consists of the amino acid sequence

(SEQ ID NO: 6)
TTDVVFRDGNGQIQSN.

In the amino acid sequence ITXGGTT (SEQ ID NO: 7) X represents a natural amino acid. In particular, X represents a natural amino acid selected from the group consisting of alanine (A), arginine (R), asparagine (N), aspartic acid (D), cysteine (C), glutamic acid (E), glutamine (Q), glycine (G), histidine (H), hydroxyproline (O), isoleucine (I), leucine (L), methionine (M), phenylalanine (F), proline (P), serine (S), threonine (T), tryptophan (W), tyrosine (Y), and valine (V).

In a preferred embodiment, X represents a natural amino acid selected from serine (S) or asparagine (N). The corresponding CDR2 comprising or consisting of the amino acid sequence ITXGGTT, wherein X is selected from serine (S) and asparagine (N), is denoted as SEQ ID NO: 8.

In a particularly preferred embodiment, X represents serine (S). The corresponding CDR2 comprises or consists of the amino acid sequence ITSGGTT (SEQ ID NO: 2).

In another particularly preferred embodiment, X represents asparagine (N). The corresponding CDR2 comprises or consists of the amino acid sequence ITNGGTT (SEQ ID NO: 5).

Therefore, in one embodiment the VHH specifically binding to plasmin comprises complementarity determining regions (CDRs) CDR1, CDR2 and CDR3, wherein:
(a) CDR1 comprises or consists of the amino acid sequence GNIFSINA (SEQ ID NO: 1) or GRRFMVVA (SEQ ID NO: 4);
(b) CDR2 comprises or consists of the amino acid sequence ITSGGTT (SEQ ID NO: 2) or ITNGGTT (SEQ ID NO: 5); and
(c) CDR3 comprises or consists of the amino acid sequence

(SEQ ID NO: 3)
NADGYYSDYDKNLAEFNS
or
(SEQ ID NO: 6)
TTDVVFRDGNGQIQSN.

In a preferred embodiment the VHH specifically binding to plasmin comprises CDR1, CDR2, and CDR3 comprising or consisting of the amino acid sequences GNIFSINA (SEQ ID NO: 1), ITSGGTT (SEQ ID NO: 2), and NADGYYSDYDKNLAEFNS (SEQ ID NO: 3), respectively.

VHH A11 of the invention, which comprises the aforementioned CDR regions, was found to specifically bind to plasmin. VHH A11 was further found to be a fibrinolysis-activating VHH.

In another preferred embodiment the VHH specifically binding to plasmin comprises CDR1, CDR2, and CDR3 comprising or consisting of the amino acid sequences GRRFMVVA (SEQ ID NO: 4), ITNGGTT (SEQ ID NO: 5), and TTDVVFRDGNGQIQSN (SEQ ID NO: 6), respectively.

VHH C5 of the invention, which comprises the aforementioned CDR regions, was found to specifically bind to plasmin. VHH C5 was further found to be a fibrinolysis-activating VHH.

The CDR sequences of exemplary VHHs of the invention are provided in the following Table 1. The CDRs comprise or consist of these sequences.

TABLE 1
CDR sequences of exemplary VHHs of the invention

| | CDR1 | CDR2 | CDR3 |
|---|---|---|---|
| VHH | (SEQ ID NO:) | (SEQ ID NO:) | (SEQ ID NO:) |
| A11 | GNIFSINA (1) | ITSGGTT (2) | NADGYYSDYDKNLAEFNS |
| | | | (3) |
| C5 | GRRFMVVA (4) | ITNGGTT (5) | TTDVVFRDGNGQIQSN |
| | | | (6) |
| Consensus | | ITXGGTT (7), | |
| sequence | | wherein | |
| | | X is any natural | |
| | | amino acid | |
| Consensus | | ITXGGTT (8), | |
| sequence | | wherein | |
| | | X is Ser or Asp | |

As detailed above, the CDRs of the VHH of the invention are flanked by framework regions FR1 to FR4 in the order FR1-CDR1-FR2-CDR2-FR3-CDR3-FR4. The respective arrangement of CDR1 to CDR3 and FR1 to FR4 constitutes the variable domain of the VHH of the invention. The VHH of the invention may further comprise a C-terminal His6-tag. The His6-tag may be linked to the variable domain of the VHH of the invention via a linker, e.g. comprising the amino acid sequence GGLPETG (SEQ ID NO: 21).

The FR sequences of exemplary VHHs of the invention are provided in the following Table 2. The FRs comprise or consist of these sequences.

TABLE 2
FR sequences of exemplary VHHs of the invention

| | FR1 | FR2 | FR3 | FR4 |
|---|---|---|---|---|
| VHH | (SEQ ID NO:) | (SEQ ID NO:) | (SEQ ID NO:) | (SEQ ID NO:) |
| A11 | QVQLVETGGG | MDWYRQAP | NYADSVKGRF | WGQGTQV |
| | LVQAGGSLRL | GKERELVAA | TISRDNAKDTV | TVSS (15) |
| | SCAAS (12) | (13) | YLQMNSLKPE | |
| | | | DAAFYYC (14) | |
| C5 | QVQLVESGGG | MGWYRQAP | NYAGSVKGRF | WGQGTQVTV |
| | LVQAGGSLRL | GNQRELVA | TISGDSAKNTV | SS (19) |
| | SCAAS (16) | S (17) | YLHMNSLKPE | |
| | | | DTAVYYC (18) | |

The variable domain sequences of exemplary VHHs of the invention with or without a C-terminal His6-tag are provided in the following Table 3. The variable domains comprise or consist of these sequences.

TABLE 3
Variable domain (V_{H}H) sequences with or without a C-terminal His6-tag
of exemplary V_{H}Hs of the invention

| | Variable domain (V_{H}H) sequence with or without a C- |
|---|---|
| VHH | terminal His6-tag (SEQ ID NO:) |
| A11 | QVQLVETGGG LVQAGGSLRL SCAASGNIFS INAMDWYRQA |
| | PGKERELVAA ITSGGTTNYA DSVKGRFTIS RDNAKDTVYL |
| | QMNSLKPEDA AFYYCNADGY YSDYDKNLAE FNSWGQGTQV |
| | TVSS (9) |
| C5 | QVQLVESGGG LVQAGGSLRL SCAASGRRFM VVAMGWYRQA |
| | PGNQRELVAS ITNGGTTNYA GSVKGRFTIS GDSAKNTVYL |
| | HMNSLKPEDT AVYYCTTDVV FRDGNGQIQS NWGQGTQVTV SS |
| | (10) |
| A11 | QVQLVETGGG LVQAGGSLRL SCAASGNIFS INAMDWYRQA |
| including C- | PGKERELVAA ITSGGTTNYA DSVKGRFTIS RDNAKDTVYL |
| terminal | QMNSLKPEDA AFYYCNADGY YSDYDKNLAE FNSWGQGTQV |
| His6-tag | TVSSGGLPETG HHHHHH (11) |
| C5 | QVQLVESGGG LVQAGGSLRL SCAASGRRFM VVAMGWYRQA |
| including C- | PGNQRELVAS ITNGGTTNYA GSVKGRFTIS GDSAKNTVYL |
| terminal | HMNSLKPEDT AVYYCTTDVV FRDGNGQIQS NWGQGTQVTV |
| His6-tag | SSGGLPETGH HHHHH (20) |

Accordingly, in a preferred embodiment, the VHH of the invention comprises or consists of the amino acid sequence of SEQ ID NO: 9 or 11; or an amino acid sequence which is at least 75%, 80%, 85%, 90%, 95%, 96%, 97%, 98%, 99%, or 100% identical to an amino acid of SEQ ID NO: 9 or SEQ ID NO: 11.

VHH A11 of the invention, which comprises the variable domain of SEQ ID NO: 9 or 11, was found to specifically bind to plasmin. VHH A11 was further found to be a fibrinolysis-activating VHH.

In another preferred embodiment the VHH of the invention comprises or consists of the amino acid sequence of SEQ ID NO: 10 or 20, or an amino acid sequence which is at least 75%, 80%, 85%, 90%, 95%, 96%, 97%, 98%, 99%, or 100% identical to an amino acid of SEQ ID NO: 10 or SEQ ID NO: 20.

VHH C5 of the invention, which comprises the variable domain of SEQ ID NO: 10 or 20, was found to specifically bind to plasmin. VHH C5 was further found to be a fibrinolysis-activating VHH.

The VHH of the invention specifically binds to plasmin, preferably to human plasmin.

It was found that VHHs A11 and C5 of the invention specifically bound to human plasmin. The binding to mouse plasmin was weak, and no reactivity to mouse plasminogen was found. Also, no binding to miniplasmin was found.

In particular, the VHH binds to human plasmin with an affinity (K_{D}) of 130 nM or less, preferably 100 nM or less, more preferably 70 nM or less, more preferably 50 nM or less, more preferably 40 nM or less, more preferably 30 nM or less, still more preferably 20 nM or less, most preferably 10 nM or less.

It was found that VHHs A11 and C5 of the invention specifically bound to human plasmin with an affinity (K_{D}) in the low nM-range.

The affinity (K_{D}) can be determined by methods known in the art. For example, affinity is determined at RT using Biolayer Interferometry (BLI) technology, e.g. as described in the examples.

Preferably, the VHH of the invention does not bind to the truncated plasmin variant ocriplasmin.

Further, the VHH of the invention binds to plasminogen with an affinity (K_{D}) of 50 nM or more, preferably 70 nM or more, more preferably 80 nM or more, more preferably 90 nM or more, most preferably 100 nM or more.

The VHH of the invention has been found to exhibit thrombolytic activity. As used herein, the term “thrombolytic activity” relates to the capacity of a substance, e.g. the VHH of the invention, to dissolve blood clots by activating plasminogen, thereby forming a cleaved product called plasmin. Methods used for the assessment of thrombolytic activity are well known in the art. Suitable methods are described in Illich A, Bokarev I, Key N S. Global assays of fibrinolysis. Int J Lab Hematol 2017; 39: 441-447.

Fibrinolysis describes the process of removing (lysing) the clot formed by activation of hemostatic pathways, either in physiological response to vascular trauma or in pathological thrombosis. The fibrinolytic system is activated either directly or indirectly by proteins that convert plasminogen to plasmin. As described above, the activation of plasminogen into plasmin is mediated by two types of activators, urokinase-type plasminogen activator (uPA) and tissue-type plasminogen activator (tPA).

The VHH of the invention has been found to exhibit fibrinolysis-activating activity, particularly in plasma or whole blood, particularly in vitro. An exemplary clot lysis model for the assessment of the fibrinolytic activity is described in the example provided below. In this model clot formation is induced in whole blood or plasma in the presence of exogenously added tPA and clot formation and clot lysis are monitored by thrombelastometry.

The VHH of the invention has further been found to accelerate plasmin generation, particularly in vitro.

For example, the plasmin generation assay can be used, as described in the examples. For example, plasmin generation in vitro is accelerated 1.2, 1.5, 2, 3, 4, 5, 6, 7, 8, 9, 10 or more fold.

The VHH of the invention has further been found to increase the catalytic efficacy of plasmin for peptide substrates. In particular, the VHH of the invention has been found to increase the conversion rate of a plasmin peptide substrate by plasmin in a dose-dependent manner, particularly in vitro.

Preferably, the assay as described in the examples can be used. In particular, to test the properties of VHHs to increase the conversion rate of a plasmin peptide substrate by plasmin, hydrolysis rates of a fluorogenic plasmin peptide substrate by plasmin were measured in the presence of increasing concentrations of VHHs and the mean velocity of substrate conversion determined.

Plasmin substrates also include activated coagulation factors such as activated factor V (FVa). Plasmin-mediated inactivation of FVa can be measured using a prothrombinase assay as described by Hamedani et al. 2020.

The VHH A11 of the invention has further been found to reduce FVa inactivation by plasmin to between about 80% to about 60% of normal, thus reaching a comparable activity to tranexamic acid (TXA), i.e. a known plasmin protector/inhibitor. As expected the direct acting plasmin inhibitor BPTI reduced FVa inactivation to 20%. The inhibitory effect of the VHHs on plasmin-induced inactivation of FVa can be explained by binding of the VHHs to an exosite of plasmin that is involved in the binding of plasmin to FVa but not in the cleavage of fibrin.

The VHH of the invention has further been found to increase tPA-induced plasmin generation, particularly in vitro, and/or to increase fibrin degradation rates, particularly in vitro.

Preferably, the assays as described in the examples can be used.

In order to assess the influence of VHHs on tPA-mediated plasminogen activation, plasminogen was preincubated with increasing concentrations of VHHs. After addition of tPA, plasmin generation was measured over time using a fluorogenic plasmin peptide substrate.

To further test the fibrinolysis-activating properties of VHHs in an ex-vivo model of hyperfibrinolysis, a whole blood based assay can be used whereat citrate-anticoagulated whole blood in the presence of VHHs is spiked with tPA and clot formation and the fibrinolysis rate of the formed clot monitored by thrombelastometry after initiation of clotting by addition of CaCl₂) solution containing recombinant TF.

Accordingly, in one embodiment the VHH of the invention specifically binding to plasmin
exhibits thrombolytic activity; and/or
exhibits fibrinolysis-activating activity in plasma or whole blood; and/or
accelerates plasmin generation in vitro; and/or
increases the conversion rate of a plasmin peptide substrate by plasmin in a dose-dependent manner; and/or
increases tPA-induced plasmin generation in vitro; and/or
increases fibrin degradation rate in vitro.

In another aspect, the invention relates to a VHH specifically binding to plasmin, wherein:
(i) plasmin is human plasmin; and/or
(ii) the VHH binds to human plasmin with an affinity (K_{D}) of 50 nM or less;
(iii) the VHH exhibits thrombolytic activity; and/or
(iv) the VHH exhibits fibrinolysis-activating activity in plasma or whole blood; and/or
(v) the VHH accelerates plasmin generation in vitro; and/or
(vi) the VHH increases the conversion rate of a plasmin peptide substrate by plasmin in a dose-dependent manner; and/or
(vii) the VHH increases tPA-induced plasmin generation in vitro; and/or
(viii) the VHH increases fibrin degradation rate in vitro.

It was found that the VHHs A11 and C5 of the invention specifically bind to human plasmin.

For this aspect, the same preferred embodiments apply as for the other aspects of the invention.

The invention further relates to a compound comprising at least one VHH of the invention.

In particular, the VHH may comprise one or more further moieties. These may be linked to the VHH covalently or non-covalently.

The compound comprising at least one VHH of the invention may further comprise one or more moieties selected from a chemical moiety providing for an extended half-life in vivo, a PEG moiety, an immunoglobulin which is not capable of specific binding to a human antigen, transferrin, human albumin, a homoamino acid polymer (HAP), a proline-alanine-serine (PAS) polymer, hydroxyethyl starch (HES), an elastin-like peptide (ELP), an Fc moiety, a peptide tag, an aptamer, a targeting moiety and an antibody or antibody fragment.

In particular, the compound comprising at least one VHH of the invention may further comprises one or more moieties covalently or non-covalently bound to the at least one VHH.

For example, the at least one VHH of the invention may be covalently linked to a chemical moiety having a molecular weight of at least 25 kDa, 35 kDa, 65 kDa, 85 kDa, or 185 KDa. For example, the molecular weight of the chemical moiety may be up to about 3000 kDa, 2000 KDa or 1000 kDa.

The one or more moieties may be polymers such as PEG (Veronese F M, 2008) or polymer mimetics such as hydrophilic and flexible polypeptide chains as used in the XTEN (Schellenberger, 2009) or PASylation technologies (Binder et al., 2017). Further preferred scaffolds increasing the half-life are carbohydrates, such as dextran, polysialic acids, hyaluronic acid, dextrin, or hydroxyethyl starch. For example, such moieties may be bound via linker, thereby providing a covalent linkage.

In another preferred embodiment of the present invention, the compound comprising at least one VHH of the invention is selected from a PEGylated VHH, VHH linked to hyaluronic acid and VHH fused to at least one peptide or protein, preferably wherein the VHH fused to at least one peptide or protein is a VHH fused to an Fc, VHH fused to XTEN, VHH fused to an immunoglobulin, preferably an antibody, which is not capable of specific binding to a human antigen, VHH fused to Transferrin, VHH fused to Albumin, preferably human serum Albumin, VHH fused to PEG, VHH fused to a homoamino acid polymer (FIAP), VHH fused to a proline-alanine-serine (PAS) polymer, VHH fused to a carbohydrate, more preferably selected from dextran, polysialic acids, hyaluronic acid, dextrin, and hydroxyethyl starch (HES), or VHH fused to a elastin-like peptide (ELP). Accordingly, in one preferred embodiment, the “one or more moiety” is selected from a PEG moiety, such as PEG50, PEG1000 or PEG2000, XTEN, hyaluronic acid, at least one peptide or protein, an immunoglobulin which is not capable of specific binding to a human antigen, an Fc, Transferrin, Albumin, recombinant PEG, a homoamino acid polymer (FIAP), a proline-alanine-serine (PAS) polymer, carbohydrate, more preferably selected from dextran, polysialic acids, hyaluronic acid, dextrin, and hydroxyethyl starch (HES), and an elastin-like peptide (ELP).

In case of covalent linkage to one or more moiety which is a protein, the VHH may be linked to the N-terminus, the C-terminus or internally, via an amino acid side chain of the one or more moiety. In case of covalent linkage to one or more moiety which is an antibody, the VHH may be linked to the C-terminus of the light chain or fragment thereof, to the C-terminus of the heavy chain or fragment thereof, to the N-terminus of the light chain or fragment thereof or to the N-terminus of the heavy chain or fragment thereof, or internally, via an amino acid side chain of heavy and/or light chain. In case of covalent linkage to an Fc molecule, the VHH may be linked to the N-terminus, the C-terminus or internally, via an amino acid side chain of the Fc protein.

The Fc molecule may be an Fc molecule having a native Fc sequence or a genetically engineered Fc molecule.

In one embodiment, the compound comprising at least one VHH of the invention is bispecific or multispecific. Optionally, the compound further comprises at least one moiety specifically binding to a blood clot antigen.

A blood clot antigen is understood as antigen which is present in a blood clot, in particular a human blood clot. The antigen may be e.g. a protein, peptide or a carbohydrate, in particular a protein.

For example, suitable clot antigens include thrombin, prothrombin, antithrombin-Ill, apolipoprotein A-I, complement components (C)3a, and C5b-C9, histidine-rich glycoprotein (HRG), proteoglycan, immunoglobulin, apolipoprotein B-100, a platelet-derived protein, fibrinogen, α2-antiplasmin, α2-macroglubulin, factor XIII and TSP1.

In a preferred embodiment, the blood clot antigen is not plasmin.

For example, variable domains or VHH domains binding to such blood clot antigens are either known in the art or can be prepared with standard procedures.

Compounds comprising at least one VHH of the invention specifically binding to plasmin which are bispecific or multispecific, such as bispecific or trispecific, can be prepared by methods known in the art.

For example, the compound may comprise 1, 2, 3, 4 or more VHHs of the invention specifically binding to plasmin and at least one, such 1, 2, 3, 4, or more moieties specifically binding to a different antigen, such as a blood clot antigen.

The one or more moieties specifically binding to a different antigen may comprise or consist of an antibody, or an antigen-binding fragment thereof, a VHH, a Chimeric Antigen Receptor (CAR) or a receptor specific for the antigen. Where the moiety is an antibody, or an antigen-binding fragment thereof, it may be, for example, a F(ab′)2, a Fab, a diabody, or an scFv. For example, the compound may be a fusion protein, such as a bispecific fusion protein.

For example, a bispecific compound may comprise a VHH of the invention specifically binding to plasmin and a further VHH specifically binding to a different antigen, which is preferably a blood clot antigen. The VHHs may be linked via a peptide linker, or directly, via a covalent peptide bond. Suitable peptide linkers are known in the art.

### Pharmaceutical Compositions

The invention further relates to a pharmaceutical composition comprising the VHH of the invention or comprising a compound comprising the VHH of the invention and a pharmaceutically acceptable carrier or excipient.

The content of the VHH or the compound in the pharmaceutical composition is not limited as far as it is useful for treatment, prevention, or amelioration but preferably contains 0.0000001-10% by weight per total composition.

Further, the VHH, or the compound described herein is/are preferably employed in one or more pharmaceutically acceptable carrier(s).

The term “carrier” describes any molecule which may improves the active agent's selectivity, effectiveness and/or safety of administration to a human or animal body, such as by continuous or triggered release or by allowing membrane permeation of the VHH or the compound.

A carrier is further considered as being pharmaceutically acceptable, when it does not have any or not substantially adverse unwanted effects on the human or animal body, e.g. it is considered generally safe, nontoxic and/or does not cause unwanted biological side reactions. Suitable pharmaceutically acceptable carriers are well known to the person skilled in the art. The choice of carrier may depend upon route of administration and concentration of the active agent(s) and the carrier may be in the form of a lyophilised composition or an aqueous solution. Generally, an appropriate amount of a pharmaceutically acceptable salt is used in the carrier to render the composition isotonic. Examples of the carrier include but are not limited to saline, Ringer's solution and dextrose solution. Preferably, acceptable excipients, carriers, or stabilisers are non-toxic at the dosages and concentrations employed, including buffers such as citrate, phosphate, and other organic acids; salt-forming counter-ions, e.g. sodium and potassium; low molecular weight (>10 amino acid residues) polypeptides; proteins, e.g. serum albumin, or gelatine; hydrophilic polymers, e.g. polyvinylpyrrolidone; amino acids such as histidine, glutamine, lysine, asparagine, arginine, or glycine; carbohydrates including glucose, mannose, or dextrins; monosaccharides; disaccharides; other sugars, e.g. sucrose, mannitol, trehalose or sorbitol; chelating agents, e.g. EDTA; non-ionic surfactants, e.g. Tween, Pluronics or polyethylene glycol; antioxidants including methionine, ascorbic acid and tocopherol; and/or preservatives, e.g. octadecyldimethylbenzyl ammonium chloride; hexamethonium chloride; benzalkonium chloride, benzethonium chloride; phenol, butyl or benzyl alcohol; alkyl parabens, e.g. methyl or propyl paraben; catechol; resorcinol; cyclohexanol; 3-pentanol; and m-cresol). Suitable carriers and their formulations are described in greater detail in Remington's Pharmaceutical Sciences, 17th ed., 1985, Mack Publishing Co.

Preferred carriers include sterile water and saline aqueous solution, such as PBS.

### Nucleic Acids, Vectors, Host Cells

In a further embodiment, the invention relates to a nucleic acid encoding the VHH of the invention.

The invention further relates to a vector comprising the nucleic acid encoding the VHH of the invention.

In another embodiment, the invention relates to a host cell comprising the nucleic acid encoding the VHH of the invention or comprising the vector comprising the nucleic acid encoding the VHH of the invention.

The term “nucleic acid” describes any form of deoxyribonucleic acid (DNA), ribonucleic acid (RNA) or artificial nucleic acid known to the person skilled in the art.

Nucleotide sequences encoding a VHH described herein, and modified versions of these VHHs can be determined using methods well known in the art, i.e., nucleotide codons known to encode particular amino acids are assembled in such a way to generate a nucleic acid that encodes the VHH. Such a polynucleotide encoding the VHH can be assembled from chemically synthesized oligonucleotides, which, briefly, involves the synthesis of overlapping oligonucleotides containing portions of the sequence encoding the VHH, annealing and ligating of those oligonucleotides, and then amplification of the ligated oligonucleotides by PCR.

When a clone containing a nucleic acid encoding a particular VHH is not available, but the sequence of the VHH molecule is known, a nucleic acid encoding the VHH can be chemically synthesized or obtained from a suitable source (e.g., cells selected to express an VHH described herein) by PCR amplification using synthetic primers hybridizable to the 3′ and 5′ ends of the sequence or by cloning using an oligonucleotide probe specific for the particular gene sequence to identify, e.g., a DNA clone that encodes the VHH. Amplified nucleic acids generated by PCR can then be cloned into replicable cloning vectors using any method well known in the art.

DNA coding for the VHHs of the invention described herein can be readily isolated and sequenced using conventional procedures (e.g., by using oligonucleotide probes that are capable of binding to sequences encoding FR1 and FR4 regions. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce VHHs or antibodies, to obtain the synthesis of the VHHs in the recombinant host cells.

To generate VHHs, PCR primers including VHH variable domain nucleotide sequences, a restriction site, and a flanking sequence to protect the restriction site can be used to amplify the VHH sequences. Utilizing cloning techniques known to those of skill in the art, the PCR amplified VHH domains can be cloned into vectors, which optionally express further domain(s) such as a purification tag, as in the examples, or a protein providing for an extended half-life in vivo. In certain embodiments, the vectors for expressing the VHH domain comprise a promoter, a secretion signal, a cloning site for the variable region, and a selection marker such as neomycin. The vectors are then transfected into cell lines to generate stable or transient cell lines that express VHHs using techniques known to those of skill in the art.

Site-directed or high-density mutagenesis of the variable region or other mutagenesis methods can be used to optimize specificity, affinity, etc. of a VHH. Especially, affinity maturation strategies are known in the art and can be employed to generate high affinity VHHs.

Preferably, the nucleic acid is part of a vector.

Generally, vectors (also expression vectors) are plasmids which are used to introduce a desired nucleic acid sequence, such as a gene, into a target cell, resulting in the transcription and translation of the protein encoded by the nucleic acid sequence, e.g. the VHH or compound of the invention. Therefore, the expression vector in general comprises regulatory sequences, such as promoter and enhancer regions, as well as a polyadenylation site in order to direct efficient transcription of the nucleic acid sequence on the expression vector. The expression vector may further comprise additional necessary or useful regions, such as a selectable marker for selection in eukaryotic or prokaryotic cells, a purification tag for the purification of the resulting protein, a multiple cloning site or an origin of replication.

Usually, the expression vector may be a viral or a non-viral vector. In general, various kinds of viral vectors, such as retroviral vectors, e.g. lentiviral or adenoviral vectors, or plasmids may be used. It is preferred that the nucleic acid is part of a vector.

Such a vector comprising the nucleic acid of the invention may further be introduced in a host cell.

Methods for introducing such a vector into a host cell are well known to the person skilled in the art, such as any known transfection method, e.g. any nonviral transfection method (e.g. chemical-based, non-chemical or particle-based) or any virus-based transfection method. Examples of suitable methods are transfection methods based on calcium phosphate precipitation, lipofection, cationic polymers, Fugene, Dendrimer, nanoparticles, microinjection, cell squeezing, electroporation, particle gun (also known as gene gun), magnet assisted transfection, optical transfection, protoplast fusion, impalafection, hydrodynamic delivery, sonoporation, transferrin-based infection, antibody-mediated transfection or virus-based transfection (e.g. based on adenoviral or lentiviral vectors).

Suitable host cells are well known to the person skilled in the art, such as mammalian cells (such as human, mouse, rat or hamster cells), insect cells, bacterial cells or yeast cells. Such host cell may comprise a nucleic acid of the present invention e.g. integrated in its genome or in a vector. Methods for introducing such nucleic acid in the host cell are described above and further well known to the person skilled in the art.

### Medical Device

In another embodiment, the invention relates to a medical device comprising the VHH of the invention or the compound comprising the VHH of the invention or the pharmaceutical composition comprising the VHH of the invention or comprising the compound comprising the VHH of the invention.

In one embodiment the VHH, compound or pharmaceutical composition is covalently or non-covalently coated onto the medical device.

In another embodiment the medical device is an implantable medical device.

In particular, the medical device is selected from a stent, a catheter or balloon catheter.

The VHHs or compounds may be incorporated into or affixed to the medical device in a number of ways and utilizing any biocompatible materials, it may be incorporated into, e.g., a polymer or a polymeric matrix and sprayed onto the outer surface of the medical device. A mixture of the VHHs or compounds and the polymeric material may be prepared in a solvent, such as water or saline, or a mixture of solvents and applied to the surfaces of the medical device, such as a stent, also by dip-coating, brush coating and/or dip/spin coating, the solvent (s) being allowed to evaporate to leave a film with entrapped VHHs or compounds. In the case of medical devices where the drug(s) is delivered from micropores, struts or channels, a solution of a polymer may additionally be applied as an outlayer to control the VHHs or compounds release. The VHHs or compounds may also be attached by a covalent bond, e.g., esters, amides or anhydrides, to the stent surface, involving chemical derivatization. The VHHs or compounds may also be incorporated into a biocompatible porous ceramic coating, e.g., a nanoporous ceramic coating.

Examples of polymeric materials include hydrophilic, hydrophobic or biocompatible biodegradable materials, e.g., polycarboxylic acids; cellulosic polymers; starch; collagen; hyaluronic acid; gelatin; lactone-based polyesters or copolyesters, e.g., polylactide; polyglycolide; polylactide-glycolide; polycaprolactone; polycaprolactone-glycolide; poly(hydroxybutyrate); poly(hydroxyvalerate); polyhydroxy(butyrate-co-valerate); polyglycolide-co-trimethylene carbonate; poly(diaxanone); polyorthoesters; polyanhydrides; polyaminoacids; polysaccharides; polyphosphoesters; polyphosphoester-urethane; polycyanoacrylates; polyphosphazenes; poly(ether-ester) copolymers, e.g., PEO-PLLA, fibrin; fibrinogen; or mixtures thereof; and biocompatible non-degrading materials, e.g., polyurethane; polyolefins; polyesters; polyamides; polycaprolactame; polyimide; polyvinyl chloride; polyvinyl methyl ether; polyvinyl alcohol or vinyl alcohol/olefin copolymers, e.g., vinyl alcohol/ethylene copolymers; polyacrylonitrile; polystyrene copolymers of vinyl monomers with olefins, e.g., styrene acrylonitrile copolymers; ethylene methyl methacrylate copolymers; polydimethylsiloxane; poly(ethylene-vinylacetate); acrylate based polymers or coplymers, e.g., polybutylmethacrylate, poly(hydroxyethyl methylmethacrylate); polyvinyl pyrrolidinone; fluorinated polymers, such as polytetrafluoethylene; cellulose esters, e.g., cellulose acetate, cellulose nitrate or cellulose propionate; or mixtures thereof.

### Medical Uses

As the VHH of the invention which specifically binds to plasmin may modulate the fibrinolytic and/or thrombolytic activity, accelerates and/or increases plasmin generation and increases fibrin degradation, it is particularly suitable for use in the treatment, prevention or amelioration of a disease or condition characterized by a thrombotic or embolic state.

Therefore, the invention further relates to the VHH of the invention for use in the treatment, prevention or amelioration of a disease or condition characterized by a thrombotic or embolic state.

Analogously, the invention also relates to the compound or the pharmaceutical composition or the medical device comprising at least one VHH of the invention for use in the treatment, prevention or amelioration of a disease or condition characterized by a thrombotic or embolic state.

In particular, the disease or condition characterized by a thrombotic or embolic state is selected from embolism, lung embolism, stroke, infarction, brain infarction, thrombotic stroke, a microthrombotic disorder, venous occlusive disease, ischemia, thrombosis in dialysis patients, acute myocardial infarction, deep vein thrombosis, acute ischemic stroke, acute peripheral arterial occlusion, occlusion of indwelling catheters, intracardiac thrombus formation and microthrombotic ischemia.

The VHH of the invention is further particularly suitable for use in emergency medicine, particularly for use in emergency lysis in patients with acute thrombotic or embolic state and risk factors for residual emboli.

For example, the VHH of the invention or the compound or pharmaceutical composition comprising the VHH of the invention can be administered to a patient in patients diagnosed with acute thrombotic or embolic state and risk factors for residual emboli or diagnosed to be at risk of having an acute thrombotic or embolic state. For example, the VHH of the invention or the compound or pharmaceutical composition comprising the VHH of the invention can be administered within 10 minutes, 20 minutes, 30 minutes, 60 minutes, 2 hours, 3, 4, 5, 6, 7, 8, 9, 10, 12 or 24 hours after being diagnosed with acute thrombotic or embolic state and risk factors for residual emboli or being diagnosed to be at risk of having an acute thrombotic or embolic state.

The VHH of the invention is further particularly suitable for use in emergency medicine in the treatment, prevention or amelioration of a disease or condition characterized by a thrombotic or embolic state, particularly selected from embolism, lung embolism, stroke, infarction, brain infarction, thrombotic stroke, a microthrombotic disorder, venous occlusive disease, ischemia, thrombosis in dialysis patients, acute myocardial infarction, deep vein thrombosis, acute ischemic stroke, acute peripheral arterial occlusion, occlusion of indwelling catheters, intracardiac thrombus formation and microthrombotic ischemia.

The VHH of the invention is further particularly suitable for use in the treatment, prevention or amelioration of a disease or condition characterized by a thrombotic or embolic state, wherein the disease or condition is an acute or chronic disease or condition.

The VHH of the invention or the compound or pharmaceutical composition comprising the VHH of the invention can be administered by any suitable route of administration including oral administration and parenteral administration such as intranasal, subcutaneous, intravenous, intraarterial, intracardial and intramuscular administration. Preferably, the VHH of the invention or the compound or pharmaceutical composition comprising the VHH of the invention is administered parenterally.

Therefore, in one embodiment, the VHH, compound or pharmaceutical composition can be formulated for parenteral administration. In a preferred embodiment, the VHH, compound or pharmaceutical composition is formulated for parenteral administration, in particular selected from intravenous, intraarterial, intracardiac, intradermal, subcutaneous, intraembolic, intramucosal or intraarticular administration. For example, the VHH, compound or pharmaceutical composition can be formulated for intravenous administration, such as intravenous infusion or injection.

In another preferred embodiment, the VHH, compound or pharmaceutical composition is administered by parenteral administration, in particular selected from intravenous, intraarterial, intracardiac, intradermal, subcutaneous, intraembolic, intramucosal or intraarticular administration. For example, the VHH, compound or pharmaceutical composition is administered by intravenous administration, such as intravenous infusion or injection.

In a further embodiment the VHH, compound, pharmaceutical composition, or medical device for use as described herein is used in combination with a second thrombolytic agent.

The second thrombolytic agent is preferably selected from a tPA protein, an uPA protein, streptokinase, alteplase, reteplase, tenecteplase, urokinase, prourokinase, and anistreplase (APSAC).

These thrombolytic agents are well-known in the art and are approved or used as thrombolytic agents.

The second thrombolytic agent can be administered to a subject at a lower dose in the combination comprising a VHH of the invention and a second thrombolytic agent as compared to the administration of the second thrombolytic agent alone. For example, the dose may be lowered by 10%, 20%, 30%, 40%, 50% or 90%.

For example, the second thrombolytic agent can be administered spatially separately with the VHH, compound or pharmaceutical composition or together, such as a single compositions, vial, container, kit or as kit-of parts. For example, the second thrombolytic agent can be administered temporally separately from the VHH, compound or pharmaceutical composition, or at the same time point, and/or within 1, 2, 3, 4, 5, 6, 7, 8, 9, 10 or 30 minutes or within 1, 2, 3, 4, 5, 6, 7, 8, 9, 10, 12, 24 or 48 hours.

The invention further relates to a method of treating, preventing or ameliorating a disease or condition characterized by a thrombotic or embolic state in a subject in need thereof, comprising administering to said subject a pharmaceutically effective amount of the VHH, compound of pharmaceutical composition.

As used herein, the term “pharmaceutically effective amount” in the context of the administration of a therapy to a subject refers to the amount of a therapy that achieves a desired therapeutic, preventive/prophylactic or ameliorating effect.

A suitable amount and dosage can be determined by persons skilled in the art. For example, a VHH or pharmaceutical composition described herein may administered to a subject (e.g., via intravenous injection) at about 0.001 mg/kg, 0.01 mg/kg 0.1 mg/kg, 0.3 mg/kg, 1 mg/kg, 3 mg/kg, 6 mg/kg, or about 10 mg/kg.

Thereby, administering of the VHH, compound or pharmaceutical composition of the present invention refers to any route of drug administration known to the person skilled in the art, such as parenteral, intravenous, intraperitoneal, subcutaneous, oral, intranasal or sublingual administration. Suitable dosage regimens are also well known to the person skilled in the art. Preferably, the VHH, compound or pharmaceutical composition of the present invention is administered in a pharmaceutically effective amount, i.e. in a dose or concentration causing a biological response in the body the VHH, compound or pharmaceutical composition is administered to.

In general, the disclosure is not limited to the particular methodology, protocols, and reagents described herein because they may vary. Further, the terminology used herein is for the purpose of describing particular embodiments only and is not intended to limit the scope of the present disclosure. As used herein and in the appended claims, the singular forms “a”, “an”, and “the” include plural reference unless the context clearly dictates otherwise. Similarly, the words “comprise”, “contain” and “encompass” are to be interpreted inclusively rather than exclusively.

“About” is understood as the mean of the indicated value±10% or ±5%.

Unless defined otherwise, all technical and scientific terms and any acronyms used herein have the same meanings as commonly understood by one of ordinary skill in the art in the field of the disclosure. Although any methods and materials similar or equivalent to those described herein can be used in the practice as presented herein, the specific methods, and materials are described herein.

The disclosure is further illustrated by the following figures and examples, although it will be understood that the figures and examples are included merely for purposes of illustration and are not intended to limit the scope of the disclosure unless otherwise specifically indicated.

### DESCRIPTION OF THE FIGURES

FIG. 1. Plasminogen activation using different concentrations of uPA and tPA. Plasma purified plasminogen (200 μg/ml) was incubated for 24 h (open symbols), 48 h (gray solid symbols), and 72 h (black solid symbols) with increasing concentrations of (A) u-PA or (B) t-PA in activation buffer containing 100 mM tranexamic acid (TXA). Formation of plasmin was monitored overtime through SDS-PAGE as described under Materials and Methods in the Example. Squares show plasminogen (PLG) amount and circles show generated plasmin (PLM) amount in percentage of total intensity of the corresponding bands on SDS-PAGE gel.

FIG. 2. VHHs competition ELISA. The VHH (A) A11 or (B) C5 was immobilized in the microtiter plate by overnight incubation of 10 μg/ml VHH in 100 μl of coating buffer. Subsequently, wells were overlaid with plasmin (10 nM) and plasmin binding to the immobilized VHHs was measured in the presence of free VHHs (A11 or C5) or TXA at the indicated concentrations in HEPES-NaCl buffer through plasmin activity testing. Data represent the mean±SD of two measurements.

FIG. 3. Effect of plasmin VHHs on the catalytic activity of plasmin for peptide substrates. Hydrolysis rates of the fluorogenic plasmin peptide substrate (H-Ala-Phe-Lys-AMC,) by plasmin (5 nM) were measured in the presence of VHHs, BPTI, TXA, or a negative control (anti-GFP VHH) at the indicated concentrations and the mean velocity of the reaction during a five minute incubation period was calculated. Mean values and SD were calculated out of three measurements.

FIG. 4. Influence of VHHs on endogenous fibrinolysis. (A) Citrate-anticoagulated whole blood was mixed with VHHs A11 or C5 and with tPA to achieve a final concentration of 0.5 μg/ml and clot formation was induced by addition of CaCl₂) (0.2 M). (B) Lysis index at 60 min(C) Maximum lysis rates, and (D) lysis onset time were recorded. Mean values and SD were calculated out of three measurements.

FIG. 5. Influence of VHHs on tPA-mediated plasminogen activation. Plasminogen (10 nM final concentration) was preincubated with increasing concentrations of VHHs (A11 or C5), BPTI or TXA for 30 min at 37° C. After addition of tPA (10 nM final concentration) plasmin generation was measured over time using the fluorogenic plasmin assay. The data are presented as mean±SD of three independent measurements.

FIG. 6. (A) Influence of VHHs on plasmin-mediated FVa cleavage. Plasmin (1.5 nM) was incubated with increasing concentrations of VHHs for 30 min at 37° C. After addition of FVa (30 pM) and further incubation for 30 min, FXa (0.52 pM), prothrombin (10 nM) and a fluorogenic thrombin substrate (100 μM) were added and the FVa-dependent thrombin generation was measured. The FVa inactivation rate measured in the absence of VHHs or plasmin inhibitors was set as 100%. (B) TXA and BPTI at increasing concentrations were used as controls. The data are presented as mean±SD of two independent measurements.

FIG. 7. Impact of VHHs binding on α₂-AP-mediated plasmin inactivation. Plasmin (10 nM) was incubated with VHHs (3.16 μM) or tranexamic acid (TXA) (3 mM) for 30 min followed by addition of 10 nM α₂AP. After 30 min of incubation, the residual activity of plasmin was measured by addition of 50 μl of the sample to 50 μl of a plasmin-specific fluorogenic substrate (400 μM). The plasmin activity in the wells in the absence of VHHs and α₂AP was considered as 100% activity and relative residual plasmin activities calculated accordingly. The data are presented as mean±SD of three independent measurements.

FIG. 8. Effect of VHHs on plasmin-induced endothelial cell barrier dysfunction. Human umbilical vein endothelial cells forming a confluent monolayer on permeability inserts of 96-culture wells were overlaid with 100 nM plasmin (PLM, A, upper panel) or a mixture of 100 nM plasminogen (PLG) and 100 nM tissue-type plasminogen activator (tPA, A, lower panel) in the absence (negative control) or presence of VHHs (A11 or C5, each 3 μM) or BPTI (30 nM). After 2 h of incubation, the endothelial cell barrier permeability was quantified by the flux of FITC-bound dextran from the upper into the lower chamber. Results are shown for PLM- (B) and for PLG/tPA-treated cells (C) and given as fold-permeability normalized to non-treated cells. Data are shown as means with standard deviations of four independent measurements. Groups were compared using ANOVA, showing that VHHs did not significantly alter PLM-activity on endothelial cell integrity * p<0.05, ** p<0.01, *** p<0.001

FIG. 9. Effect of tranexamic acid and lysine on the stability of plasmin. (A) Time-dependent degradation of plasmin was measured in buffer containing 100 mM TXA or lysine. (B) Influence of incubation temperature tPA on PLM stability.

FIG. 10. The impact of VHHs on the clot formation parameters in plasma monitored by rotational thrombelastometry. Citrate-anticoagulated pooled normal human plasma was mixed with VHHs, BPTI (2.4 μM), or TXA (2.4 μg/ml). After addition of tPA to reach a final concentration of 0.1 μg/ml, coagulation was initiated by addition of recombinant tissue factor (Dade® Innovin®) dissolved 1:100 in calcium chloride solution (0.2 M). The viscoelasticity of the formed clot was measured in terms of (A) clotting time, (B) maximum clot firmness, and (C) maximum clot elasticity. A non-binder VHH (GFP-enhancer VHH) was used as negative control. Mean values and SD were calculated out of three measurements.

The invention is further described by way of the following example which is not to be construed as limiting the scope of the invention.

### EXAMPLES

### Plasmin Kringle Domain Binding VHHs Enhance the Fibrinolytic Activity

The fibrinolytic system controls intravascular clot formation and is critically involved in cellular remodeling including wound healing through proteolytic degradation of fibrin and extracellular matrix components by plasmin.

The objective of the study was to determine whether plasmin VHHs (i.e. high-affinity plasmin binding VHHs) may modulate the fibrinolytic activity.

Therefore, wild-type plasmin that was generated and purified from plasma-purified plasminogen was used to immunize alpacas and generate a VHH-library. Binding characteristics of plasmin binding hit compounds were analyzed and high affinity plasmin binders characterized by a variety of assays including blood-based and cell-based assays.

### Materials and Methods

### Materials

All basic chemicals as well as SYBR green, sepharose 4B, cyanogen bromide, L-lysine monohydrochloride, human uPA, and the in vitro vascular permeability assay were purchased from Merck/Sigma-Aldrich (Darmstadt, Germany). Aprotinin (BPTI) was bought from Applichem (Darmstadt, Germany). TXA was purchased from Carino Pharma (Elze, Germany). Plasma derived human Glu-plasminogen, human Lys-plasmin which is prepared from homogeneous Glu-plasminogen using uPA, mouse Glu-plasminogen, mouse plasmin, plasma-derived human activated factor V, human activated factor X, human prothrombin and human α₂-AP were purchased from Haematologic Technologies, Inc. (Essex Junction, VT). The fluorogenic peptide substrates for plasmin (H-Ala-Phe-Lys-AMC) and thrombin (Boc-Asp(OBzl)-Pro-Arg-AMC) were purchased from Bachem (Bubendorf, Switzerland). The Berichrom® plasminogen assay kit and recombinant human tissue factor (Dade® Innovin®) were obtained from Siemens Healthcare Diagnostics (Marburg, Germany). Recombinant tPA (Actilyse) was purchased from Boehringer Ingelheim (Biberach, Germany). Coomassie brilliant blue R-250 staining and destaining solutions, 2× laemmli sample buffer, and mini-PROTEAN® TGX™ precast polyacrylamide gel were purchased from Bio-Rad Laboratories (Munich, Germany). PageRuler™ prestained protein ladder, the SilverXpress® silver staining kit, Pierce™ BCA protein assay kit, and Gibco™ trypsin-EDTA (0.05%) were purchased from thermo fisher scientific (Waltham, US). Human umbilical vein endothelial cells (HUVECs), endothelial cell basal medium (ECBM), endothelial cell growth medium (ECGM), and phenol red-free ECBM were purchased from Promocell (Heidelberg, Germany).

### Plasminogen and Plasmin Activity Assays

The concentration of plasminogen was measured using a commercially available peptide substrate assay (Berichrom plasminogen, Siemens Healthcare, Germany). Samples to be analyzed were diluted 1:1 and 1:3 (vol/vol) in assay buffer (10 mM HEPES, 154 mM NaCl, 1 mg/ml BSA, pH 7.4) and 2 μl of diluted samples were mixed with 100 μl of streptokinase reagent. After incubation at 37° C. for 5 min 10 μl of the chromogenic substrate (HD-Nva-CHA-Lys-pNA, 3 mM) was added and substrate cleavage rates were monitored by kinetic measurements at 405 nm using the Synergy 2 microplate reader. A standard curve was prepared by serial dilution of commercially available plasminogen (Hematologic Technologies Inc., Essex Junction, VT).

Plasmin activity was measured through hydrolysis rates of the fluorogenic peptide substrate (H-Ala-Phe-Lys-AMC, Bachem (Bubendorf, Switzerland). Samples to be analyzed were diluted in assay buffer (10 mM Tris-HCl, 154 mM NaCl, 1 mM MgCl₂, 1 mM CaCl₂, 0.1% BSA) and 50 μl of the diluted samples were transferred to the wells of black F16 Fluoronunc modules (Thermo Fisher Scientific) and mixed with 50 μl of the plasmin fluorogenic peptide substrate to reach a final concentration of 400 μM. Subsequently, substrate hydrolysis rates were monitored using the Synergy 2 microplate reader.

### Plasminogen Purification

Plasminogen was purified from human plasma by affinity chromatography using L-lysine substituted sepharose and subsequent fast protein liquid chromatography (FPLC). Lysine affinity chromatography was performed as described (Summaria et al 1976, Deutsch and Mertz 1970). Pooled human citrate-anticoagulated plasma was diluted 1:1 (vol/vol) with 0.9% NaCl containing 6 mM EDTA. A volume of 350 ml-500 ml of diluted plasma was passed through the column with a flow rate of 75 ml/h. The column was washed with 300 ml washing buffer (0.3 M NaH₂PO₄/Na₂HPO₄-buffer pH 7.4) at 1.25 ml/min flow rate until the absorbance at 280 nm of collected fractions was less than 0.01. Plasminogen was eluted with elution buffer (0.1 M NaH₂PO₄/Na₂HPO₄-buffer containing 0.1 M ε-aminocaproic acid or 0.1 M lysine, pH 7.4) at a flow rate of 1.6 ml/min. The protein concentration of each fraction was determined at 280 nm and the fractions showing an optical density of 0.1 or higher were analyzed by sodium-dodecyl-sulfate polyacrylamide-gel-electrophoresis (SDS-PAGE) followed by silver staining. Plasminogen-containing fractions as tested using the plasminogen activity test were pooled and the volume reduced by filtration using ultra centrifugal filter units (Amicon®, Merck/Sigma-Aldrich, Darmstadt, Germany) with 50 kDa nominal molecular weight limit. To achieve highly pure plasminogen, a further purification step using FPLC equipped with a HiPrep 16/60 Sephacryl S-300 column (Cytiva, Freiburg, Germany) with a separation range of 10-150 kDa, was used. The column was equilibrated with equilibration buffer (10 mM HEPES, 154 mM NaCl, pH 7.4) followed by injection and loading of 5 ml of the plasminogen sample in the same buffer through the sample loop. The flow rate was set to 0.5 ml/min and fraction collection was performed every two minutes. Fractions showing a plasminogen-protein ratio>0.9 were pooled, concentrated by filtration and aliquots stored at −80° C. until used.

### Immunization of Alpacas and VHHs Production

Two alpacas (Vicugna pacos) were subcutaneously immunized 6× using 200 μg uPA-activated plasmin in HEPES buffer (10 mM HEPES, 154 mM NaCl, 100 mM tranexamic acid, pH 7.4) mixed 1:1 with GERBU-FAMA adjuvants over the time course of 12 weeks. After the immunization step, 100 ml blood was drawn and peripheral blood mononuclear cells (PBMCs) were isolated by centrifugation on a Ficoll gradient. The RNA content of PBMCs was extracted subsequent to cell lysis. Reversely transcribed cDNA is then cloned in phagemid-vector and used for solid-phase panning. Biotinylated plasmin was loaded on streptavidin beads and incubated with phagemids to capture plasmin-specific VHHs. The captured sequences were subsequently eluted using elution buffer (0.2 M glycine, pH 2.2). The hit-compounds were expressed and subjected to BugSup-ELISA to quantify the binding affinity of VHHs to plasminogen and plasmin.

### Binding Studies

VHHs binding affinities to human and mouse plasminogen/plasmin were assessed according to Biolayer Interferometry (BLI) using the BLItz system (Pall Life Sciences, Dreieich, Germany) and the Blitz 1.2 software package (Hamedani 2016). A high-affinity anti-HIS antibody-coated biosensor (HIS2) (Pall Life Sciences) was loaded for 2 min with 0.1 mg/ml HIS-tagged VHHs. Then, for determination of association rate constants (ka and kₒₙ), the loaded biosensor was equilibrated in binding buffer (20 mM HEPES, 154 mM NaCl, 0.1% BSA) for 2 min and subsequently lowered into a drop that contained plasminogen or plasmin in binding buffer. Afterward, the biosensor was lowered into a tube containing 500 μl of binding buffer to determine kd and k_{off}. All measurements were performed at a shaking speed of 2,200 rpm.

To assess the competition of VHHs for similar or overlapping binding sites on the plasmin molecule, a competition ELISA was performed. Microtiter (96-well) plates were coated with VHHs (10 μg/ml in coating buffer, 100 μl/well) at 2-8° C. overnight. Wells were washed and non-specific protein-binding sites were blocked with 20 mg/ml bovine serum albumine (BSA) for 2 h at RT. After removing the blocking buffer, wells were overlaid with a reaction mixture containing plasmin (10 nM) and VHHs (0.316 nM-31.6 μM) in buffer (20 mM HEPES, 154 mM NaCl, BSA 1 mg/ml, pH 7.4) for 30 min at RT. After washing the amount of bound plasmin was quantified through hydrolysis rates of the fluorogenic plasmin substrate (300 μM, 100 μl/well). The fluorescence intensity measured in the absence of free VHHs was set as 100%.

Influence of VHHs on Catalytic Activity of Plasmin with Peptide Substrates

Plasmin (20 nM) was incubated in assay buffer (10 mM Tris-HCl, 154 mM NaCl, 1 mM MgCl₂, 1 mM CaCl₂, 1 mg/ml BSA, pH 7.4) with increasing concentrations of each VHH (20 μM to 200 μM) or the plasmin inhibitors BPTI (20 μM to 20 μM) or TXA (20 nM to 300 mM) for 30 min at 37° C. After incubation, 50 μl of the mixtures were transferred to the wells of black F16 Fluoronunc modules (Thermo Fisher Scientific [Nunc]). Subsequently, 50 μl of 800 μM PLM-specific fluorogenic peptide substrate in the assay buffer (H-Ala-Phe-Lys-AMC, Bachem) was added to the wells and the kinetics of plasmin-mediated substrate hydrolysis was monitored using the Synergy 2 microplate reader.

To analyse Michaels-Menten kinetics in the presence of VHHs, plasmin (5 nM) was incubated with VHHs (1 μM), BPTI (3 nM) or TXA (50 mM) for 30 min in a total volume of 50 μl of assay buffer. Subsequently, 50 μl of the fluorogenic substrate (3.16-3,160 μM final concentration in assay buffer) was added. Substrate hydrolysis rates were monitored by kinetic measurements using the Synergy 2 microplate reader. Apparent Michaelis-Menten constants (Kₘ) and the turn-over number (k_{cat}) values for substrate hydrolysis were calculated from best fit values of the Michaelis-Menten equation and the catalytic efficiencies were expressed as ratios of k_{cat}/Kₘ.

### Influence of VHHs on the Fibrinolytic Activity in Plasma and Whole Blood

The impact of VHHs on the fibrinolytic activity in plasma and whole blood was monitored using rotational thrombelastometry using the ROTEM delta system (TEM Innovations Ltd., Munich, Germany). Citrate-anticoagulated pooled normal human plasma (CPP, in-house preparation) was mixed with VHHs, TXA, or BPTI. After addition of 0.1 μg/ml tPA, coagulation was initiated by addition of recombinant tissue factor (Dade Innovin, Siemens Healthcare, Marburg, Germany) dissolved 1:100 in calcium chloride (0.2 M). The viscoelasticity of the formed clot was measured in terms of key parameters including clotting time, maximal clot firmness, maximum clot elasticity, maximum lysis, lysis index after 60 minutes, and lysis onset time.

### Plasmin Generation Assay

Plasminogen (50 nM) was incubated with VHHs (2 μM to 0.2 μM) for 30 min at 37° C. in assay buffer. Subsequently, 20 μl of 50 nM tPA was added to the mixture and incubated at 37° C. After 3 hours of incubation at 37° C., 50 μl of the mixture were transferred to the wells of black F16 Fluoronunc modules and after addition of 50 μl of the plasmin-specific fluorogenic substrate (800 μM) fluorescence intensities were measured at λₑₓ of 360 nm and λem of 460 nm using the Synergy 2 microplate reader. BPTI (1 pM to 1 μM) and TXA (1 nM to 1 mM) were used as positive controls.

### Activated Factor V-Inactivation Assay

Plasmin-mediated inactivation of activated factor V (FVa) was measured using a prothrombinase assay as recently described (Hamedani 2020). Briefly, 1.5 nM plasmin in assay buffer (20 mM Tris-HCl, 137 mM NaCl, 10 mg/ml phospholipids, 5 mM CaCl₂, 1 mg/ml BSA, pH 7.4) was preincubated with VHHs (0.1 nM-10 μM final concentrations) for 30 min at 37° C. Then, 60 μl of the mixture were mixed with 60 μl of 300 μM FVa. After further incubation for 30 min, 25 μl of the reaction mixture were mixed with 50 μl of 1.3 μM human FXa and 100 μM fluorogenic thrombin substrate (Boc-Asp(OBzl)-Pro-Arg-AMC) and 50 μl of 10 nM human prothrombin. Thrombin catalyzed substrate hydrolysis was monitored at λₑₓ of 360 nm and λₑₘ of 460 nm using the Synergy 2 microplate reader. BPTI (1 μM to 10 μM) and TXA (0.03 mM to 100 mM) were used as controls.

### Plasmin Inhibition Assay

Human plasmin (10 nM) was incubated with VHHs (3.16 μM), or TXA (3 mM) for 30 min at 37° C. and then α₂-AP (10 nM) added. After incubation for 30 min at 37° C., 50 μl of the mixture were transferred to the wells of black F16 Fluoronunc modules and 50 μl of plasmin-specific fluorogenic substrate (800 μM) were added and substrate hydrolysis measured at λₑₓ of 360 nm and λₑₘ of 460 nm. The plasmin activity in the reactions containing all reagents except α₂-AP was set as 100%.

### Endothelial Barrier Permeability Assay

The permeability assay was performed as previously described with minor changes (Hamedani 2020). Briefly, HUVECs of passages 5 to 8 were seeded at a density of 2.5×10⁴ cells/well into collagen-coated permeability inserts of 96-well culture plates (Merck, Darmstadt, Germany) and cultured in ECGM (125 μl upper chamber, 250 μl lower chamber) until reaching confluency. One hour before the experiment, ECGM was replaced with phenol red-free ECBM. Plasmin (100 nM) was incubated with VHHs (3 μM final concentrations) or BPTI (30 nM final concentration) for 30 min in phenol red-free ECBM. In a parallel experiment, plasminogen (100 nM) was incubated first with tPA (100 nM) for 30 min followed by incubation of the mixture with VHHs or aprotinin with the same concentration for another 30 min. The mixture was added to the upper chamber of the permeability insert and after 2 h of incubation the medium in the upper chamber was replaced with FITC-Dextran diluted in phenol red-free ECBM and the lower chamber medium was replaced with 250 μl of the phenol red-free ECBM. After 30 min of incubation at RT under light protection, the relative fluorescence intensity of 100 μl of the medium of the lower chamber was measured at λex of 485 nm and λem of 535 nm using a Synergy 2 microplate reader. The fluorescence intensity in the wells treated with plasmin or plasminogen-tPA was defined as 100% flux of FITC-bound dextrane.

### Preparation of Lysine Sepharose

Sepharose 4B beads (40 ml) were washed and resuspended in 0.1 M NaHCO₃ buffer, pH 9.0, followed by addition of 4 g CNBr and pH adjustment to 11 for carboxylic group activation. Eight grams of L-lysine monohydrochloride was dissolved in 20 ml of 0.1 M NaHCO₃-buffer and added to the washed sepharose beads. To reach the maximum loading of lysine on Sepharose beads, the slurry was stirred overnight at 4° C. The day after, the Sepharose beads were settled down and a sub-sample of 100 μl from of the supernatant was taken. The loading efficiency of lysine on Sepharose beads was quantified from the initial and residual amount of lysine in slurry supernatant using a ninhydrin-based assay.

In order to pack a XK-16/40 column (GE Healthcare), 30 ml of lysine-sepharose was packed to the column. The column was equilibrated at RT using 0.1 M NaH₂PO₄/Na₂HPO₄-buffer containing 3 mM EDTA (pH 7.4) and stored at 2 to 8° C. in the same buffer containing 0.02% w/v sodium azide for further use.

Column purification of plasminogen was done at room temperature. To avoid clot formation, 3 mM EDTA was added to all buffers.

### Amino Acid and Protein Concentration Determination Using Ninhydrin and Bradford Assays

In order to quantify lysine concentration, 200 μl of sample was mixed with 50 μl of ninhydrine in acetone (8%, w/v) and incubated at 80° C. and 400 rpm shaking speed for 15 min. Samples were then cooled down followed by addition of 50 μl of 50% ethanol (v/v in water). The absorbance was measured in duplicate at 570 nm using an automated Synergy 2 microplate reader (BioTek, Bad Friedrichshall, Germany). The Bradford assay was performed as previously described in the art.

### SDS-Polyacrylamide Gel Electrophoresis (SDS-PAGE)

SDS-PAGE was performed using a Modular Mini-Protein II electrophoresis system (Bio-Rad, Munich, Germany) followed by Coomassie Blue or silver staining to analyze the purity and molecular mass of the proteins. Ten microliter of protein solution (0.2 mg/ml for Coomassie Blue, 0.05 mg/ml for silver staining) were mixed with 11 μl of 2× Laemmli-buffer with or w/o 10% R-mercaptoethanol (R-ME), incubated for 5 min at 99° C., 350 rpm shaking speed, and electrophoresed in a 4% stacking gel and 12% resolving gel of polyacrylamide, followed by staining with 0.25% Coomassie Blue (Bio-Rad,) or by silver staining (SilverXpress™, ThermoFisher Scientific, Darmstadt, Germany).

To evaluate the purity and molecular mass of the plasminogen and plasmin purity, SDS-PAGE combined with Coomassie- or silver-staining was performed. Commercially available plasminogen and plasmin was used as standard samples. Ten microliter of plasminogen [0.2 mg/ml for Coomassie, 0.05 mg/ml for silver staining] Coomassie-staining was done for 1 h on a shaker incubator, followed by 3× de-staining using 100 ml de-staining solution (20 ml methanol, 10 ml acetic acid, 80 ml H₂O). Silver-staining was done as described in the user's manual.

### Results

### Plasmin Generation

Plasminogen was purified from normal human plasma using lysine-affinity chromatography and subsequent size-exclusion chromatography reaching a yield of 8.86% (considering 35 mg plasminogen in 175 ml plasma) and a purity of 90% (according to the band intensity resulted from Coomassie-staining of corresponding plasminogen bands on SDS-PAGE gel). Details of the purification process are summarized in the following Table 4.

TABLE 4
Purification of plasminogen from plasma

| | Total | | Specific | |
|---|---|---|---|---|
| | protein | Plasminogen | activity | Purification |
| Purification step | (mg) | activity (U) | (U/mg) | fold |
| Plasma (175 ml) | 12250 | 1.13 | 9 × 10⁻⁵ | n.a.* |
| Lysine | 11.3 | 48.6 | 4.3 | 4.8 × 10⁴ |
| chromatography | | | | |
| Amicon filtration | 9.7 | 45.6 | 4.7 | n.a. |
| Size exclusion | 3.1 | 18 | 5.8 | 6.4 × 10⁴ |
| chromatography | | | | |

*n.a. not applicable

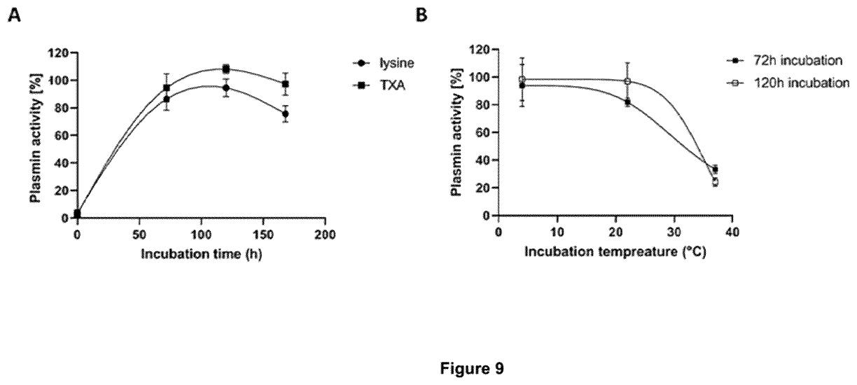

To develop a plasmin generation protocol that achieves a high plasmin yield at minimal concentrations of plasminogen activators, type and concentration of the plasminogen activator and the reaction times were systematically varied. Moreover, to avoid autocatalytic degradation of plasmin, a buffer is required that protects plasmin from autocatalytic degradation. This was achieved by addition of lysine or TXA to the activation buffer (FIG. 9A). Since TXA was more effective than lysine, TXA at a final concentration of 100 mM was used in all further experiments as plasmin protector. In addition, the impact of incubation temperature and incubation time on the activity of generated plasmin was assessed at two different incubation times and three different incubation temperatures. The optimum plasmin generation and activity was observed after 72 h at 8° C. or 22° C. (FIG. 9B)

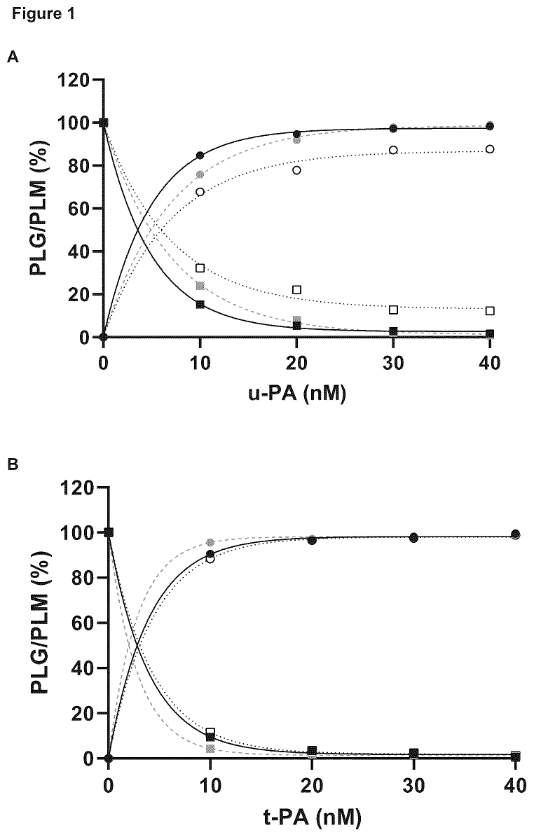

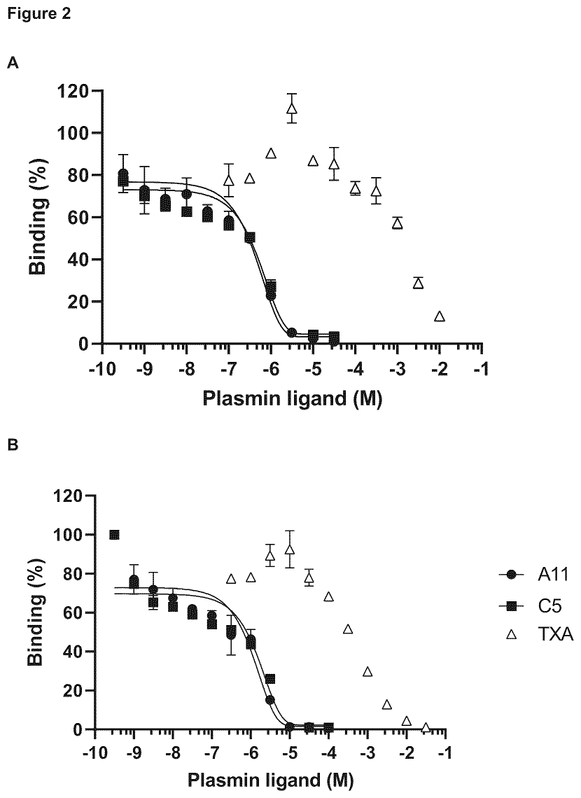

Nearly complete conversion of plasminogen to plasmin was achieved at a molar ratio of tPA or uPA to plasminogen of 1:38 or 1:23, respectively, and an incubation time of 72 h (FIG. 1). uPA was selected for plasmin generation, because its molecular weight of 49,000 allows subsequent separation from plasmin (MW: 83,000) by size exclusion chromatography. Plasmin prepared in this way showed a purity of >95% and was found stable in TXA-storage buffer for 72 h at RT.

### Plasmin VHHs are High Affinity Binders

After two rounds of enrichment 8 VHHs strains were identified showing high-affinity binding to plasminogen/plasmin. Among these, the VHHs A11 and C5 bind human plasmin with binding affinities in the low nanomolar range and mouse plasmin with affinities of 87.3 nM or 172 nM, respectively (Table 5). A11 and C5 are selective plasmin binders (K_{D} value for human PLG>100 nM). None of these VHHs bind to miniplasmin (ocriplasmin). The VHHs showed no reactivity with mouse plasminogen. C5 and A11 showed weak binding to tPA (Table 5).

Competitive binding studies demonstrated that both A11 and C5 competed for the same or overlapping binding site on plasmin molecule. TXA competed with both A11 and C5 to bind to free plasmin albeit at different level. The TXA concentration which inhibits 50% of free plasmin molecules to bind to coated A11 was 1 mM while this value for C5 was 0.46 mM (FIGS. 2A and 2B)

TABLE 5
Binding of VHHs to plasminogen and plasmin

| | k_{D} (nM) | | | | | |
|---|---|---|---|---|---|---|
| | human | human | mouse | mouse | Ocri- | |
| VHHs | plasminogen | plasmin | plasminogen | plasmin | plasmin | tPA |
| A11 | >100 | 4.7 | >1,000 | 87.3 | n.b.* | 941 |
| C5 | >100 | 6.8 | >1,000 | 172 | n.b. | 213 |

*n.b., no binding

ELISA-based competition experiments revealed that both VHHs, A11 and C5 obviously share the same binding site (FIGS. 2A and 2B).

VHHs Increase the Plasmin Catalytic Function with Small Substrates

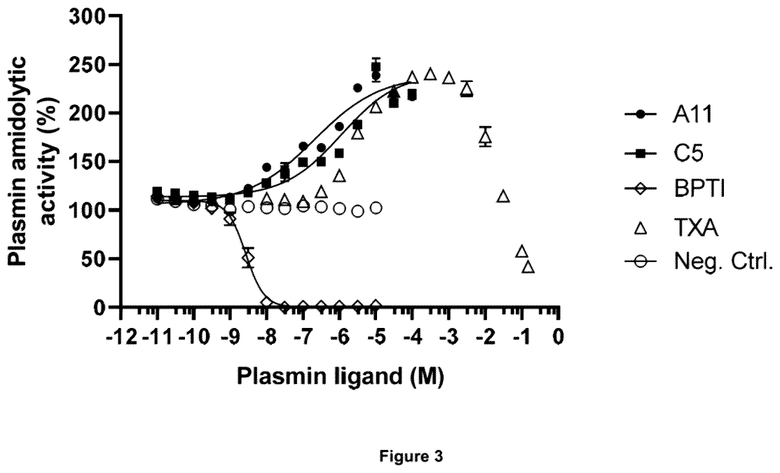

In the presence of the plasmin binding VHHs, the conversion rate of the plasmin peptide substrates is increased in a dose-dependent manner (FIG. 3). Comparable to TXA an approx. 2.5-fold increase in substrate conversion was achieved at molar ratios of VHHs over plasmin of 158 and 500 for A11 and C5, respectively. To further characterize this effect Michaelis-Menten kinetics were determined (Table 6). The two VHHs significantly increased kcat-values while Km remained stable. The direct acting plasmin inhibitor BPTI reduced the kcat-value while TXA also decreased kcat but significantly increased Kₘ.

TABLE 6
Effect of VHHs on kinetic constants of plasmin-mediated
cleavages of fluorogenic peptide substrate

| Plasmin | Kₘ (μM) | k_{cat} × 10³ (s⁻¹) | K_{cat}/Kₘ |
|---|---|---|---|
| ligand | mean (95% CI) | mean (95% CI) | (μM⁻¹ · s⁻¹) |
| w/o | 319.5 (302.7-337.2) | 51.8 (51-53) | 162 |
| A11 | 295.2 (261.4-333.3) | 67.2 (65-70) | 227.6 |
| C5 | 286.9 (263.2-312.8) | 69.9 (68-72) | 243.8 |
| BPTI | 352.1 (331.0-374.5) | 29.6 (29-30) | 84.1 |
| TXA | 563.3 (506.5-626.7) | 42.1 (40-44) | 74.8 |

Steady-state hydrolysis of the fluorogenic peptide substrate (H-Ala-Phe-Lys-AMC, 3.16-3,160 μM) by plasmin (5 nM) in the presence of VHHs (1 μM), BPTI (3 nM), and TXA (7.86 mg/ml) was measured as described in the Materials and Methods section. CI, confidence interval. ^{a} The turnover number (k_{cat}) was calculated using Vₘₐₓ values extracted from Michaelis-Menten least squares fit.

### Plasmin VHHs Upregulate Plasmin Generation

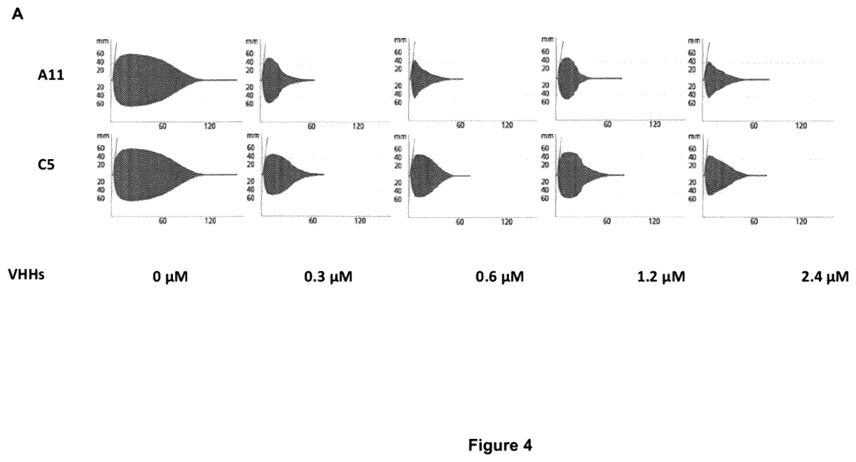

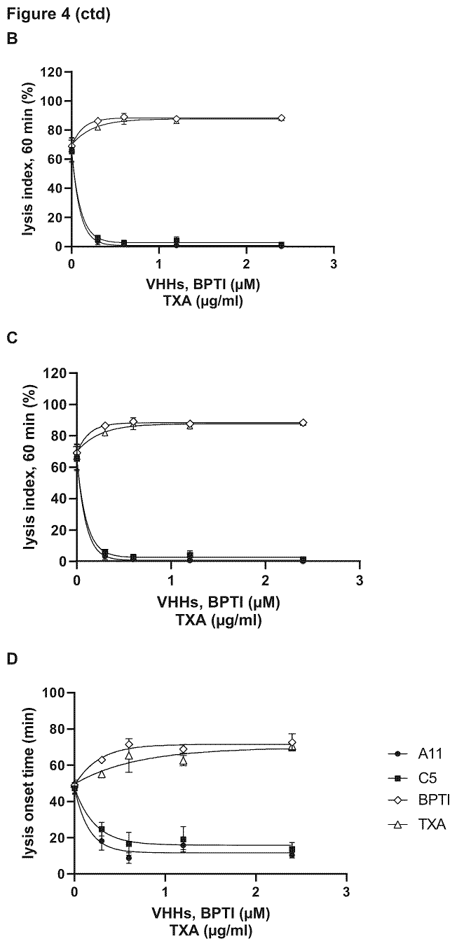

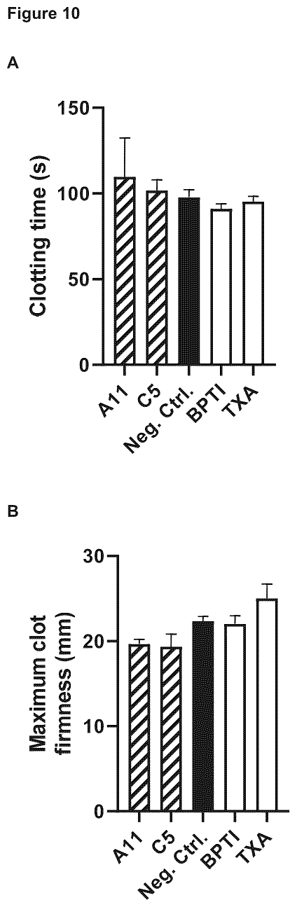

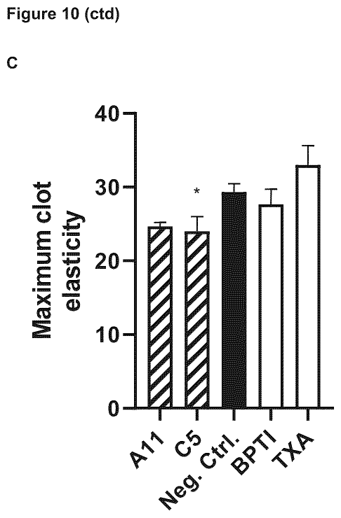

As shown in FIG. 4, the VHHs C5 and A11 induced a profibrinolytic response (FIG. 4A) with a decrease of the lysis index at 60 min from 65% to 0% at concentrations of A11 and C5 above 1.2 μM (FIG. 4B). The lysis onset time was reduced from 47.2 min to 10.3 min and 13.8 min by using A11 and C5 VHHs, respectively (FIG. 4D). As shown in FIG. 4C, both, BPTI and TXA showed opposite effects (inhibition of lysis) when compared to the assessed VHHs. In addition, spiking the citrated pool plasma with high concentration of VHHs or BPTI (2.4 μM), or TXA (2.4 μg/ml) does not affect the parameters correspond to fibrin clot formation such as clotting time, maximum clot firmness and maximum clot elasticity (FIG. 10).

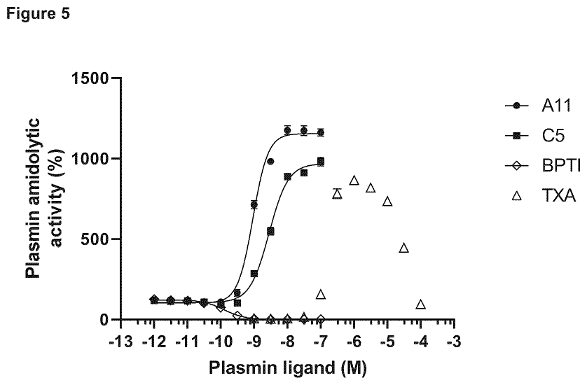

To further investigate the effect of A11 and C5 on fibrinolysis activation, tPA induced plasmin generation was studied in a purified system. Both VHHs increased tPA-induced plasmin generation in a dose-dependent manner reaching a 10-15-fold increase in the activation rate at plasma levels above 10⁻⁷ M (FIG. 5). An EC50 of 2.3 nM has been calculated for C5 while the EC50 calculated for A11 is 0.93 nM. The increase is comparable with the increase seen with TXA albeit on a higher level. Moreover, while at higher concentrations of TXA plasmin generation is blocked, A11 and C5 showed no inhibitory effect at the assessed concentrations.

A11 and C5 Protect Activated Factor V from Plasmin Proteolysis

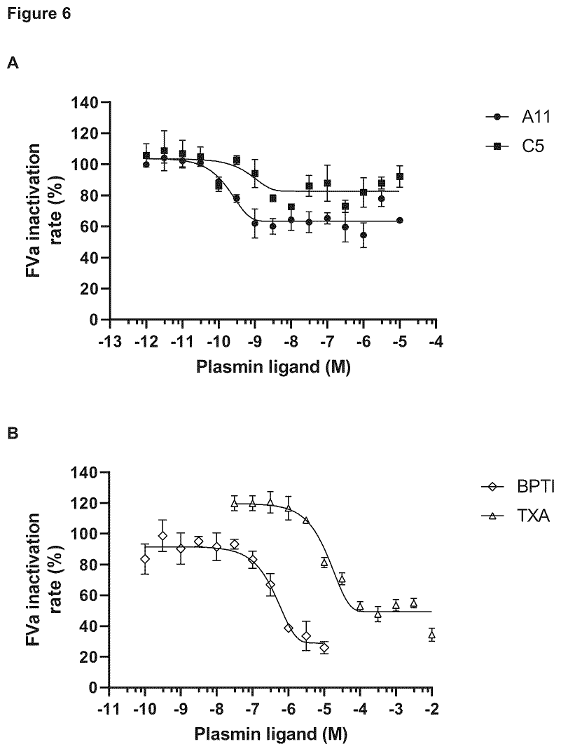

The direct acting plasmin inhibitor BPTI dose-dependently inhibits the plasmin-mediated cleavage of FVa reaching a maximal inhibition rate of 26% at a concentration of 10 μM (FIG. 6B). Compared to BPTI, the plasmin inhibiting capacity of TXA is lower reaching a maximal rate of inhibition of 48% at a concentration of 316 μM or higher. At lower TXA concentrations, FVa cleavage was found to be accelerated (FIG. 6B). The VHHs A11 and C5 significantly decreased FVa inactivation rates (FIG. 6A). At concentrations above 10-9 M corresponding to a molar ratio of 1.5 between plasmin and A11, FVa inactivation rate was reduced to 60% by the VHH A11, while the C5 concentration of 3.16 nM or higher could reduce the FVa inactivation rate to 78%. (FIG. 6B).

### Plasmin-Inhibiting Activity of Alpha2-Antiplasmin is not Influenced by Plasmin VHHs

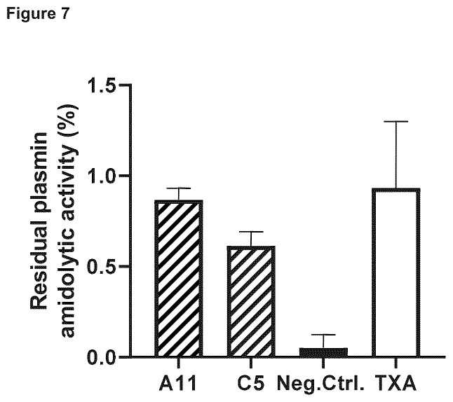

Using a purified system, inactivation rates of plasmin by the fast acting inhibitor α2AP were investigated. At a molar excess of α2AP over plasmin, a nearly complete inhibition of plasmin is achieved as indicated by a residual plasmin activity<0.1% (FIG. 7). Addition of the VHHs A11 or C5 or TXA only marginally increased the residual activity to 0.8-1%.

### Plasmin-Induced Endothelial Cell Barrier Dysfunction is not Enhanced by A11 or C5

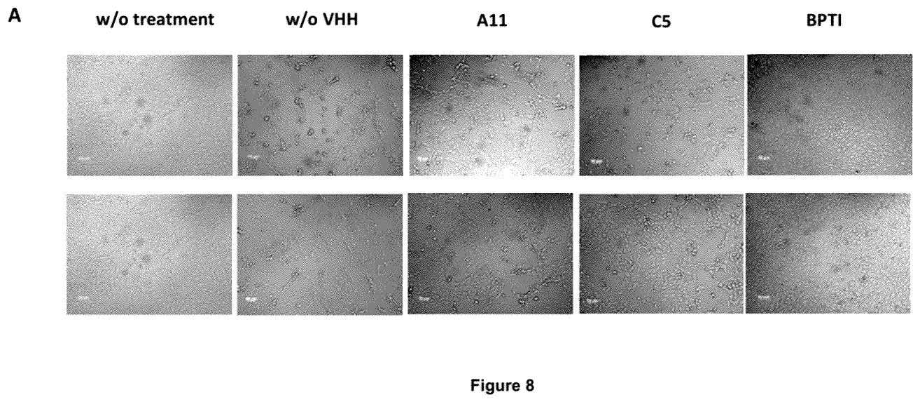

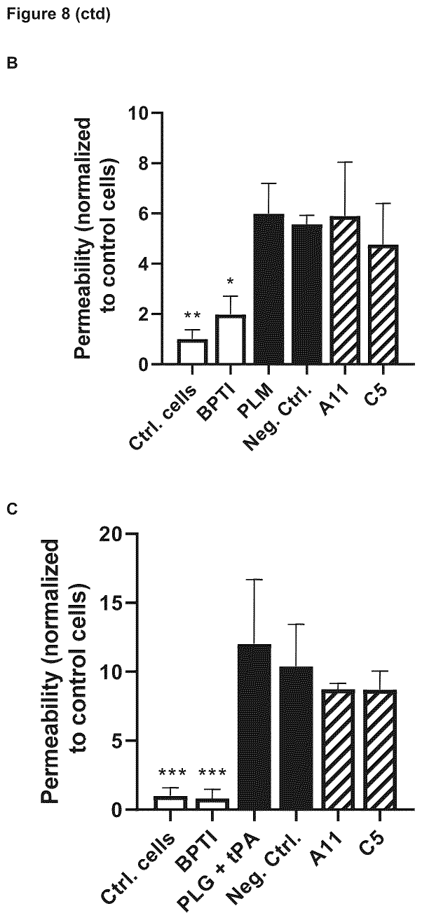

Incubation of a HUVEC-monolayer with plasmin disrupts the endothelial barrier function as indicated by an increase of albumin permeability (FIG. 8). None of the VHHs significantly decreased or increased this plasmin effect, whereas the direct acting plasmin inhibitor BPTI significantly protects the endothelial cell barrier function from plasmin destruction (FIGS. 8A and 8B). Substitution of plasmin by a tPA-plasminogen mixture results in a slightly more pronounced destruction of the endothelial cell monolayer (FIGS. 8A and 8C). In the presence of BPTI this effect is completely neutralized. The VHHs A11 and C5 neither protect nor accelerate the permeability increasing effect of tPA-plasminogen.

### DISCUSSION

High affinity plasmin binding VHHs are described that increase the activity of the plasminogen-plasmin system.

Immunization of alpacas with the TXA-stabilized plasmin antigen results in the identification of eight hit compounds showing binding to plasmin in the low nanomolar range. None of the hit compounds showed reactivity with the truncated plasmin variant ocriplasmin suggesting that the binding site of the VHHs is located outside the catalytic domain. This assumption is further supported by the results of the peptide substrate assays. The VHHs significantly increase the catalytic efficacy of plasmin for peptide substrates. This suggests that VHHs binding induces an intramolecular change of plasmin thereby increasing the availability of the active centre for peptide substrates. Conformational changes of plasmin through ligand binding that increase the catalytic activity have been reported for kringle-domain binding ligands, such as TXA, and for streptokinase (Lämmle 1980)

To study how VHHs-binding influences the interaction of plasmin with macromolecular substrates a variety of functional assays using purified components and plasma or whole blood were used. A clot lysis model was used to study the effect of the hit compounds on the fibrinolytic activity. In this model clot formation is induced in whole blood or plasma in the presence of exogenously added tPA and clot formation and clot lysis are monitored by thrombelastometry. The compounds C5 and A11 significantly increase the fibrinolytic response. At VHH plasma levels of 1 μM the lysis onset time was reduced from approx. 45 min to less than 20 min.

Theoretically, VHHs can modulate the activity level of the fibrinolytic pathway through interference with the plasminogen activation complex, interference with the reactivity of plasmin with macromolecular substrates, and interference with the inactivation rate of the generated plasmin. To study the effect of VHHs on tPA induced plasmin generation a purified system has been used. The compounds C5 and A11 increase tPA-dependent plasmin generation reaching a plateau at concentrations exceeding 10⁻⁸ M. Possible explanations how C5 and A11 accelerate tPA induced plasmin generation include a molecular change in the plasminogen structure that increases the tPA sensitivity of plasminogen. In view of the low binding affinity of both VHHs to plasminogen this explanation is unlikely. Since the VHHs showed a moderate binding affinity to tPA, one might speculate that in the presence of A11 and C5 binding and subsequent cleavage of plasminogen is enhanced. A third explanation could be, that VHHs complexed plasmin becomes a more effective autocatalytic activator of plasminogen.

Beyond fibrin, activated coagulation factors, such as FVa, are plasmin substrates. To investigate the influence of VHHs on FVa inactivation rates, a prothrombinase assay was used. C5 reduced FVa inactivation by plasmin to 80% of normal and A11 to 60% of normal reaching a comparable activity to TXA. As expected the direct acting plasmin inhibitor BPTI reduced FVa inactivation to 20%.

Increasing or lowering the rate of plasmin inactivation by α2-AP might induce an antifibrinolytic or profibrinolytic response. Inactivation rates of plasmin by plasma-purified α2-AP were only marginally influenced by A11 and C5. A similar result was observed with TXA. Since the active centre and the surrounding area are mainly involved in complex formation with α2-AP, these results support our conclusion that none of the VHHs directly bind to the active centre or a molecular structure surrounding the active centre.

Plasmin and tPA formed during the inflammation process increase the permeability of the endothelial cell lining. A variety of cellular receptors including the plasminogen receptor and Toll-like receptors are involved in this reaction. The results obtained with the endothelial cell barrier assay demonstrate that only the direct acting plasmin inhibitor BPTI significantly reduced the endothelial cell damaging activity of plasmin whereas none of the VHHs significantly reduced the cytotoxic activity of plasmin. A11 and C5 do not increase the endothelial cell permeability after plasmin exposure. In the in-vivo situation plasmin is generated from plasminogen through plasminogen activators that are released from damaged endothelial cells. To better simulate the in-vivo situation, plasmin was replaced by a mixture of tPA and plasminogen. Also, in this experimental setting A11 and C5 did not increase the damage rate of endothelial cells.

Taken together high affinity plasmin binding VHHs were established by immunization of alpacas with TXA-stabilized plasmin that was generated from plasma-purified plasminogen by uPA activation. All VHHs bind plasmin with high affinity in the low nanomolar range and increase the catalytic activity of plasmin for peptide substrates suggesting that VHHs binding induces a conformational change of plasmin that increases the availability of the active centre for small substrates. A11 and C5 increase plasmin formation. Since the increase in plasmin formation improves thrombolysis, these VHHs are compounds that can be used to make thrombolysis faster in life-threatening thrombosis such as lung-embolisms or ischemic brain infarction. The protection of FVa from plasmin degradation by FAN should reduce the risk of bleeding.

### REFERENCES

Anglés-Cano E. Overview on fibrinolysis: plasminogen activation pathways on fibrin and cell surfaces. Chem Phys Lipids. 1994 January; 67-68:353-62. doi: 10.1016/0009-3084(94)90157-0. PMID: 8187235.
Baker S K, Strickland S. A critical role for plasminogen in inflammation. J Exp Med. 2020 Apr. 6; 217(4):e20191865. doi: 10.1084/jem.20191865.
Binder U, Skerra A, PASylation®: A versatile technology to extend drug delivery, Current Opinion in Colloid & Interface Science. 2017 September; 31:10-17. ISSN 1359-0294, https://doi.org/10.1016/j.cocis.2017.06.004.
Cao Y, Chen A, An S S, Ji R W, Davidson D, Llinás M. Kringle 5 of plasminogen is a novel inhibitor of endothelial cell growth. J Biol Chem. 1997 Sep. 5; 272(36):22924-8. doi: 10.1074/jbc.272.36.22924.
Cao Y, Cao R, Veitonmski N. Kringle structures and antiangiogenesis. Curr Med Chem Anticancer Agents. 2002 November; 2(6):667-81. doi:10.2174/1568011023353705.
Castellino F J, McCance S G. The kringle domains of human plasminogen. Ciba Found Symp. 1997; 212:46-60; discussion 60-5. doi:10.1002/9780470515457.ch4.
English H, Hong J, Ho M. “NAR single domain antibody sequences, phage libraries and potential clinical applications”. Antibody Therapeutics. 2020 January; 3 (1): 1-9.
Ghannam A, Kumari S, Muyldermans S, Abbady A Q. “Camelid nanobodies with high affinity for broad bean mottle virus: a possible promising tool to immunomodulate plant resistance against viruses”. Plant Molecular Biology. 2015 March; 87 (4-5): 355-69.
Hamedani N S, Müller J, Tolle F, Rühl H, Pezeshkpoor B, Liphardt K, Oldenburg J, Mayer G, Pötzsch B. “Selective modulation of the protease activated protein C using exosite inhibiting aptamers”. Nucleic Acid Therapeutics 2020 June; 30:5, 276-288.
Hamedani N S, Rühl H, Zimmermann J J, Heiseler T, Oldenburg J, Mayer G, Pötzsch B, Müller J. In vitro evaluation of aptamer-based reversible inhibition of anticoagulant activated protein C as a novel supportive hemostatic approach. Nucleic Acid Ther. 2016 December; 26(6):355-362. doi: 10.1089/nat.2016.0645.
Harmsen M M, De Haard H J. “Properties, production, and applications of camelid single-domain antibody fragments”. Applied Microbiology and Biotechnology. 2007 November; 77 (1): 13-22.
Ilich A, Bokarev I, Key N S. Global assays of fibrinolysis. Int J Lab Hematol. 2017 October; 39(5):441-447.
Ji W R, Barrientos L G, Llinás M, Gray H, Villarreal X, DeFord M E, Castellino F J, Kramer R A, Trail P A. Selective inhibition by kringle 5 of human plasminogen on endothelial cell migration, an important process in angiogenesis. Biochem Biophys Res Commun. 1998 Jun. 18; 247(2):414-9. doi: 10.1006/bbrc.1998.8825.
Lämmle B, Duckert F. Different assessment of plasmin with different substrates. In vitro alteration of plasmin, influence of epsilon-aminocaproic acid and tranexamic acid upon its activity. Thromb Haemost. 1980 Jun. 18; 43(2):112-7.
Möller A, Pion E, Narayan V, Ball K L. “Intracellular activation of interferon regulatory factor-1 by nanobodies to the multifunctional (Mf1) domain”. The Journal of Biological Chemistry. 2010 December; 285 (49): 38348-61.
Ponting C P, Marshall J M, Cederholm-Williams S A. Plasminogen: a structural review. Blood Coagul Fibrinolysis. 1992 October; 3(5):605-14. PMID:1333289.
Schellenberger V, Wang C W, Geething N C, Spink B J, Campbell A, To W, Scholle M D, Yin Y, Yao Y, Bogin O, Cleland J L, Silverman J, Stemmer W P. A recombinant polypeptide extends the in vivo half-life of peptides and proteins in a tunable manner. Nat Biotechnol. 2009 December; 27(12):1186-90. doi: 10.1038/nbt.1588.
Tharp A C, Laha M, Panizzi P, Thompson M W, Fuentes-Prior P, Bock P E. Plasminogen substrate recognition by the streptokinase-plasminogen catalytic complex is facilitated by Arg253, Lys256, and Lys257 in the streptokinase betadomain and kringle 5 of the substrate. J Biol Chem. 2009 Jul. 17; 284(29):19511-21. doi: 10.1074/jbc.M109.005512.
Veronese F M, Mero A, Pasut G (2009). Protein PEGylation, basic science and biological applications. In: Veronese, F. M. (eds) PEGylated Protein Drugs: Basic Science and Clinical Applications. Milestones in Drug Therapy. Birkhsuser Basel. https://doi.org/10.1007/978-3-7643-8679-5_2.
Wu, Guojie; Quek, Adam J.; Caradoc-Davies, Tom T.; Ekkel, Sue M.; Mazzitelli, Blake; Whisstock, James C.; Law, Ruby H. P. “Structural studies of plasmin inhibition”. Biochemical Society Transactions. 2019 March; 47 (2): 541-557.
Anglés-Cano E. Overview on fibrinolysis: plasminogen activation pathways on fibrin and cell surfaces. Chem Phys Lipids. 1994 January; 67-68:353-62. doi: 10.1016/0009-3084(94)90157-0. PMID: 8187235.
Baker S K, Strickland S. A critical role for plasminogen in inflammation. J Exp Med. 2020 Apr. 6; 217(4):e20191865. doi: 10.1084/jem.20191865.
Calapai G, Gangemi S, Mannucci C, Minciullo P L, Casciaro M, Calapai F, Righi M, Navarra M. Systematic review of tranexamic acid adverse reactions. J Pharmacovigilance 2015; 3: 171. Doi:10.4172/2329-6887.
Cao Y, Chen A, An S S, Ji R W, Davidson D, Llinás M. Kringle 5 of plasminogen is a novel inhibitor of endothelial cell growth. J Biol Chem. 1997 Sep. 5; 272(36):22924-8. doi: 10.1074/jbc.272.36.22924.
Cao Y, Cao R, Veitonmski N. Kringle structures and antiangiogenesis. Curr Med Chem Anticancer Agents. 2002 November; 2(6):667-81. doi:10.2174/1568011023353705.
Castellino F J, McCance S G. The kringle domains of human plasminogen. Ciba Found Symp. 1997; 212:46-60; discussion 60-5. doi:10.1002/9780470515457.ch4.
Ji W R, Barrientos L G, Llinás M, Gray H, Villarreal X, DeFord M E, Castellino F J, Kramer R A, Trail P A. Selective inhibition by kringle 5 of human plasminogen on endothelial cell migration, an important process in angiogenesis. Biochem Biophys Res Commun. 1998 Jun. 18; 247(2):414-9. doi: 10.1006/bbrc.1998.8825.
Kwaan H C, Weiss I, Tallman M S. The Role of Abnormal Hemostasis and Fibrinolysis in Morbidity and Mortality of Acute Promyelocytic Leukemia. Semin Thromb Hemost. 2019 September; 45(6):612-621. doi: 10.1055/s-0039-1693478.
Picetti R, Shakur-Still H, Medcalf R L, Standing J F, Roberts I. What concentration of tranexamic acid is needed to inhibit fibrinolysis? A systematic review of pharmacodynamics studies. Blood Coagulation and Fibrinolysis 2019; 30: 1-10
Pilbrant A, Schannong M, Vessman J. Pharmacokinetics and bioavailability of tranexamic acid. Eur J Clin Pharmacol 1981; 20: 65-72
Steinmetzer T, Pilgram O, Wenzel B M, Wiedemeyer S J A. Fibrinolysis Inhibitors: Potential Drugs for the Treatment and Prevention of Bleeding. J Med Chem. 2020 Feb. 27; 63(4):1445-1472. doi: 10.1021/acs.jmedchem.9b01060.
Tharp A C, Laha M, Panizzi P, Thompson M W, Fuentes-Prior P, Bock P E. Plasminogen substrate recognition by the streptokinase-plasminogen catalytic complex is facilitated by Arg253, Lys256, and Lys257 in the streptokinase betadomain and kringle 5 of the substrate. J Biol Chem. 2009 Jul. 17; 284(29):19511-21. doi: 10.1074/jbc.M109.005512.
Zhao T, Houng A, Reed G L. Termination of bleeding by a specific, anticatalytic antibody against plasmin. J Thromb Haemost 2019; 17: 1461-1469
Shi G Y, Wu H L. Differential autolysis of human plasmin at various pH levels. Thromb Res 1988; 51: 355-364.
Franchini M, Mannucci P M. Primary hyperfibrinolysis: Facts and fancies. Thromb Res. 2018 June; 166:71-75. doi: 10.1016/j.thromres.2018.04.010. Epub 2018 Apr. 12. PMID: 29665524.
Ponting C P, Marshall J M, Cederholm-Williams S A. Plasminogen: a structural review. Blood Coagul Fibrinolysis. 1992 October; 3(5):605-14. PMID:1333289.
Haire G, Egan K, Parmar K, McKinnon T, Monteith C, O'Connor H, Kevane B, Maguire P, Szklanna P B, Galligan M, Donnelly J C, Allen S, McCallion N, Hunt B, Áinle F N. Alterations in fibrin formation and fibrinolysis in early onset-preeclampsia: Association with disease severity. Eur J Obstet Gynecol Reprod Biol. 2019 October; 241:19-23. doi: 10.1016/j.ejogrb.2019.07.035. Epub 2019 Jul. 25. PMID: 31415952.
Wu X, Benov A, Darlington D N, Keesee J D, Liu B, Cap A P. Effect of tranexamic acid administration on acute traumatic coagulopathy in rats with polytrauma and hemorrhage. PLoS One. 2019 Oct. 3; 14(10):e0223406. doi: 10.1371/journal.pone.0223406. PMID: 31581265; PMCID: PMC6776384.
Schmidt A E, Vadivel K, Whitelegge J, Bajaj S P. Plasmin-mediated proteolysis of human factor IXa in the presence of calcium/phospholipid: Conversion of procoagulant factor IXa to a fibrinolytic enhancer. J Thromb Haemost. 2020 May; 18(5):1171-1182. doi: 10.1111/jth.14773. Epub 2020 Mar. 30. PMID: 32073726.
Jain S, Acharya S S. Inherited disorders of the fibrinolytic pathway. Transfus Apher Sci. 2019 October; 58(5):572-577. doi: 10.1016/j.transci.2019.08.007. Epub 2019 Aug. 8. PMID: 31427261.
Baker S K, Chen Z L, Norris E H, Strickland S. Plasminogen mediates communication between the peripheral and central immune systems during systemic immune challenge with lipopolysaccharide. J Neuroinflammation. 2019 Aug. 28; 16(1):172. doi: 10.1186/s12974-019-1560-y. PMID: 31462325; PMCID: PMC6712655.
Shiraishi Y, Kimura A, Matsuo O, Sakata Y, Takeshita K, Ohmori T. Short-term inhibition of fibrinolytic system restores locomotor function after spinal cord injury in mice. Sci Rep. 2019 Nov. 5; 9(1):16024. doi: 10.1038/s41598-019-52621-8. PMID: 31690812; PMCID: PMC6831600.
Valente-Acosta B, Flores-Garcia M, González-Zárate G, Gerson-Cwilich R, Maldonado-Méndez M, Juárez-Vega G, Anglés-Cano E, Peña-Díaz A. Fibrinolytic Activity of Circulating Microvesicles Is Associated with Progression of Breast Cancer. Tohoku J Exp Med. 2020 February; 250(2):121-128. doi: 10.1620/tjem.250.121. PMID: 32115494.
Marcos-Contreras O A, Martinez de Lizarrondo S, Bardou I, Orset C, Pruvost M, Anfray A, Frigout Y, Hommet Y, Lebouvier L, Montaner J, Vivien D, Gauberti M. Hyperfibrinolysis increases blood-brain barrier permeability by a plasmin- and bradykinin-dependent mechanism. Blood. 2016 Nov. 17; 128(20):2423-2434. doi: 10.1182/blood-2016-03-705384. Epub 2016 Aug. 16. PMID: 27531677.
Alves N J, Kline J A. Comparative study on the inhibition of plasmin and delta-plasmin via benzamidine derivatives. Biochem Biophys Res Commun. 2015 Feb. 13; 457(3):358-62. doi: 10.1016/j.bbrc.2014.12.117. Epub 2015 Jan. 8. PMID: 25576865.
Church W R, Messier T L, Ouellette L A, Potts S E. A kringle-specific monoclonal antibody. Hybridoma. 1994 October; 13(5):423-9. doi: 10.1089/hyb.1994.13.423. PMID: 7860098.
Christen M T, Frank P, Schaller J, Llinás M. Human plasminogen kringle 3: solution structure, functional insights, phylogenetic landscape. Biochemistry. 2010 Aug. 24; 49(33):7131-50. doi: 10.1021/bi100687f. PMID: 20617841.
Zhang D, Kaufman P L, Gao G, Saunders R A, Ma J X. Intravitreal injection of plasminogen kringle 5, an endogenous angiogenic inhibitor, arrests retinal neovascularization in rats. Diabetologia. 2001 June; 44(6):757-65.doi:10.1007/s001250051685. PMID: 11440369.
Wang M, Zajicek J, Geiger J H, Prorok M, Castellino F J. Solution structure of the complex of VEK-30 and plasminogen kringle 2. J Struct Biol. 2010 March; 169(3):349-59. doi: 10.1016/j.jsb.2009.09.011. Epub 2009 Sep. 30. PMID:19800007; PMCID: PMC2826548.
Jain S, Acharya S S. Inherited disorders of the fibrinolytic pathway. Transfus Apher Sci. 2019 October; 58(5):572-577. doi: 10.1016/j.transci.2019.08.007. Epub 2019 Aug. 8. PMID: 31427261.
Mican J, Toul M, Bednar D, Damborsky J. Structural Biology and Protein Engineering of Thrombolytics. Comput Struct Biotechnol J. 2019 Jul. 2; 17:917-938. doi: 10.1016/j.csbj.2019.06.023. PMID: 31360331; PMCID: PMC6637190.
Singh S, Houng A K, Reed G L. Venous stasis-induced fibrinolysis prevents thrombosis in mice: role of α2-antiplasmin. Blood. 2019 Sep. 19; 134(12):970-978. doi: 10.1182/blood.2019000049. Epub 2019 Aug. 8. PMID: 31395599; PMCID: PMC6753621.
Doherty T M, Kelley A. Bleeding Disorders. 2019 Jun. 16. In: StatPearls [Internet]. Treasure Island (F L): StatPearls Publishing; 2020 January-. PMID: 31082094.
Castellino F J, Beals J M. The genetic relationships between the kringle domains of human plasminogen, prothrombin, tissue plasminogen activator, urokinase, and coagulation factor XII. J Mol Evol. 1987; 26(4):358-69. doi:10.1007/BF02101155. PMID: 3131537.
Hughes A L. Modes of evolution in the protease and kringle domains of the plasminogen-prothrombin family. Mol Phylogenet Evol. 2000 March; 14(3):469-78. doi: 10.1006/mpev.1999.0685. PMID: 10712851.
Chang P C, Chang Y J, Wu H L, Chang C W, Lin C I, Wang W C, Shi G Y. Mutation of human plasminogen kringle 1-5 enhances anti-angiogenic action via increased interaction with integrin alpha(v)beta(3). Thromb Haemost. 2008 April; 99(4):729-38. doi: 10.1160/TH07-06-0403. PMID: 18392331.
Koyanagi K, Narasaki R, Yamamichi S, Suzuki E, Hasumi K. Mechanism of the action of SMTP-7, a novel small-molecule modulator of plasminogen activation. Blood Coagul Fibrinolysis. 2014 June; 25(4):316-21. doi:10.1097/MBC.0000000000000032. PMID: 24784315.
Hidaka K, Gohda K, Teno N, Wanaka K, Tsuda Y. Active site-directed plasmin inhibitors: Extension on the P2 residue. Bioorg Med Chem. 2016 Feb. 15; 24(4):545-53. doi: 10.1016/j.bmc.2015.12.009. Epub 2015 Dec. 8. PMID: 26732532.
Muhunthan K, Balakumar S, Navaratnaraja T S, Premakrishna S, Arulkumaran S. Plasma concentrations of tranexamic acid in postpartum women after oral administration. Obstet Gynecol 2020; 135: 945-948
Draxler D F, Daglas M, Fernando A, Hanafi G, McCutcheon F, Ho H, Galle A, Gregory J, Larsson P, Keragala C, Wright D K, Tavancheh E, Au A E, Niego B, Wilson K, Plebanski M, Sashindranath M, Medcalf R L. Tranexamic acid modulates the cellular immune profile after traumatic brain injury in mice without hyperfibrinolysis. J Thromb Haemost. 2019 December; 17(12):2174-2187. doi: 10.1111/jth.14603. Epub 2019 Sep. 3. PMID: 31393041.
Draxler D F, Awad M M, Hanafi G, Daglas M, Ho H, Keragala C, Galle A, Roquilly A, Lyras D, Sashindranath M, Medcalf R L. Tranexamic Acid Influences the Immune Response, but not Bacterial Clearance in a Model of Post-Traumatic Brain Injury Pneumonia. J Neurotrauma. 2019 Dec. 1; 36(23):3297-3308. doi: 10.1089/neu.2018.6030. Epub 2019 Aug. 2. PMID: 31140372.
Schoergenhofer C, Buchtele N, Gelbenegger G, Derhaschnig U, Firbas C, Kovacevic K D, Schwameis M, Wohlfarth P, Rabitsch W, Jilma B. Defibrotide enhances fibrinolysis in human endotoxemia—a randomized, double blind, crossover trial in healthy volunteers. Sci Rep. 2019 Jul. 31; 9(1):11136. doi: 10.1038/s41598-019-47630-6. PMID: 31366975; PMCID: PMC6668569.
Schol-Gelok S, de Maat M P M, Biedermann J S, van Gelder T, Leebeek F W G, Lijfering W M, van der Meer F J M, Rijken D C, Versmissen J, Kruip M J H A. Rosuvastatin use increases plasma fibrinolytic potential: a randomised clinical trial. Br J Haematol. 2020 Apr. 16. doi: 10.1111/bjh.16648. Epub ahead of print. PMID: 32301122.
Fernandes L G V, Fernandes R S, Nascimento ALTO, Leite L C C. A Modified ELISA Method to Evaluate the Interaction of Schistosoma mansoni Proteins with Plasminogen. Methods Mol Biol. 2020; 2151:185-195. doi: 10.1007/978-1-0716-0635-3_15. PMID: 32452005.
Wang L, Dorn P, Zeinali S, Froment L, Berezowska S, Kocher G J, Alves M P, Brugger M, Esteves B I O, Blank F, Wotzkow C, Steiner S, Amacker M, Peng R W, Marti T M, Guenat O T, Bode P K, Moehrlen U, Schmid R A, Hall S R R. CD90+ CD146+ identifies a pulmonary mesenchymal cell subtype with both immune modulatory and perivascular-like function in postnatal human lung. Am J Physiol Lung Cell Mol Physiol. 2020 Apr. 1; 318(4):L813-L830. doi: 10.1152/ajplung.00146.2019. Epub 2020 Feb. 19. PMID: 32073879.
Gonias S L, Zampieri C. Plasminogen Receptors in Human Malignancies: Effects on Prognosis and Feasibility as Targets for Drug Development. Curr Drug Targets. 2019 Nov. 21. doi: 10.2174/1389450120666191122101658. Epub ahead of print. PMID: 31755385.
Yepes M. Urokinase-type plasminogen activator is a modulator of synaptic plasticity in the central nervous system: implications for neurorepair in the ischemic brain. Neural Regen Res. 2020 April; 15(4):620-624. doi: 10.4103/1673-5374.266904. PMID: 31638083; PMCID: PMC6975136.
Vago J P, Sugimoto M A, Lima K M, Negreiros-Lima G L, Baik N, Teixeira M M, Perretti M, Parmer R J, Miles L A, Sousa L P. Plasminogen and the Plasminogen Receptor, Plasminogen-R<sub>KT</sub>, Regulate Macrophage Phenotypic, and Functional Changes. Front Immunol. 2019 Jun. 28; 10:1458. doi: 10.3389/fimmu.2019.01458. PMID: 31316511; PMCID: PMC6611080.
Disharoon D, Marr D W M, Neeves K B. Engineered microparticles and nanoparticles for fibrinolysis. J Thromb Haemost. 2019 December; 17(12):2004-2015. doi: 10.1111/jth.14637. Epub 2019 Oct. 7. PMID: 31529593; PMCID: PMC6893081.
Morishima Y, Kamisato C, Honda Y. Combined effect of a direct oral anticoagulant edoxaban and an inhibitor of activated thrombin-activatable fibrinolysis inhibitor on clot lysis. J Thromb Thrombolysis. 2020 January; 49(1):94-99. doi: 10.1007/s11239-019-01929-3. PMID: 31396790.
Wu G, Mazzitelli B A, Quek A J, Veldman M J, Conroy P J, Caradoc-Davies T T, Ooms L M, Tuck K L, Schoenecker J G, Whisstock J C, Law R H (March 2019). “Tranexamic acid is an active site inhibitor of urokinase plasminogen activator”. Blood Advances 2019; 3: 729-733. doi:10.1182/bloodadvances.2018025429. PMC 6418500. PMID 30814058.
Tarandovskiy I D, Rajabi A A, Karnaukhova E, Buehler P W. Contradictory to its effects on thrombin, C1-inhibitor reduces plasmin generation in the presence of thrombomodulin. J Thromb Thrombolysis. 2019 July; 48(1):81-87. doi: 10.1007/s11239-019-01869-y. PMID: 31030323.
Saes J L, Schols S E M, Betbadal K F, van Geffen M, Verbeek-Knobbe K, Gupta S, Hardesty B M, Shapiro A D, van Heerde W L. Thrombin and plasmin generation in patients with plasminogen or plasminogen activator inhibitor type 1 deficiency. Haemophilia. 2019 November; 25(6):1073-1082. doi: 10.1111/hae.13842. Epub 2019 Aug. 30. PMID: 31469483; PMCID: PMC6899449.
Semeraro F, Ammollo C T, Caironi P, Masson S, Latini R, Panigada M, Pesenti A, Semeraro N, Gattinoni L, Colucci M. D-dimer corrected for thrombin and plasmin generation is a strong predictor of mortality in patients with sepsis. Blood Transfus. 2019 Nov. 19:1-8. doi: 10.2450/2019.0175-19. Epub ahead of print. PMID: 31855152.
Ji H L, Zhao R, Matalon S, Matthay M A. Elevated Plasmin(ogen) as a Common Risk Factor for COVID-19 Susceptibility. Physiol Rev. 2020 Jul. 1; 100(3):1065-1075. doi: 10.1152/physrev.00013.2020. Epub 2020 Mar. 27. PMID: 32216698; PMCID: PMC7191627.
Chen Q, Shou W, Wu W, Wang G, Cui W. Performance evaluation of thrombomodulin, thrombin-antithrombin complex, plasmin-α2-antiplasmin complex, and t-PA: PAI-1 complex. J Clin Lab Anal. 2019 July; 33(6):e22913. doi: 10.1002/jcla.22913. Epub 2019 May 15. PMID: 31090232; PMCID: PMC6642299.
Ishihara T, Nogami K, Ochi S, Ishida T, Kosaka Y, Sawada A, Inoue M, Osone S, Imamura T, Hosoi H, Shima M. Disordered hemostasis associated with severely depressed fibrinolysis demonstrated using a simultaneous thrombin and plasmin generation assay during L-asparaginase induction therapy in pediatric acute lymphoblastic leukemia. Pediatr Blood Cancer. 2020 January; 67(1):e28016. doi: 10.1002/pbc.28016. Epub 2019 Sep. 25. PMID: 31556233.
Barbosa da Cruz D, Helms J, Aquino L R, Stiel L, Cougourdan L, Broussard C, Chafey P, Riès-Kautt M, Meziani F, Toti F, Gaussem P, Anglés-Cano E. DNA-bound elastase of neutrophil extracellular traps degrades plasminogen, reduces plasmin formation, and decreases fibrinolysis: proof of concept in septic shock plasma. FASEB J. 2019 December; 33(12):14270-14280. doi: 10.1096/fj.201901363RRR. Epub 2019 Nov. 4. PMID: 31682515.
Sillen M, Weeks S, Strelkov S, Declerck P. Structural insights into the mechansims of a nanobody that stabilizes PAI-1 and modulates its activity. International Journal of Molecular Sciences 2020; 21: 5859

1. A VHH specifically binding to plasmin wherein:
(i) plasmin is human plasmin;
(ii) the VHH binds to human plasmin with an affinity (KD) of 50 nM or less;
(iii) the VHH exhibits thrombolytic activity;
(iv) the VHH exhibits fibrinolysis-activating activity in plasma or whole blood;
(v) the VHH accelerates plasmin generation in vitro;
(vi) the VHH increases the conversion rate of a plasmin peptide substrate by plasmin in a dose-dependent manner;
(vii) the VHH increase tPA-induced plasmin generation in vitro; and/or
(viii) the VHH increases fibrin degradation rate in vitro.

2. A VHH specifically binding to plasmin comprising complementarity determining regions (CDRs) CDR1, CDR2 and CDR3, wherein:
(a) CDR1 comprises the amino acid sequence GNIFSINA (SEQ ID NO: 1) or
(SEQ ID NO: 4)
GRRFMVVA;
(b) CDR2 comprises the amino acid sequence ITXGGTT (SEQ ID NO: 7) wherein X is a natural amino acid; and
(c) CDR3 comprises the amino acid sequence NADGYYSDYDKNLAEFNS (SEQ ID NO: 3) or TTDVVFRDGNGQIQSN (SEQ ID NO: 6).

3. The VHH of claim 2, wherein the CDR1, CDR2, and CDR3 comprise the amino acid sequences GNIFSINA (SEQ ID NO: 1), ITSGGTT (SEQ ID NO: 2), and NADGYYSDYDKNLAEFNS (SEQ ID NO: 3), respectively, or wherein the CDR1, CDR2, and CDR3 comprise the amino acid sequences GRRFMVVA (SEQ ID NO: 4), ITNGGTT (SEQ ID NO: 5), and TTDVVFRDGNGQIQSN (SEQ ID NO: 6), respectively.

4. The VHH of claim 2, comprising or consisting of:
(a) the amino acid sequence of SEQ ID NO: 9 or 11; or an amino acid sequence which is at least 75%, 80%, 85%, 90%, 95%, 96%, 97%, 98%, 99%, or 100% identical to an amino acid of SEQ ID NO: 9 or SEQ ID NO: 11; or
(b) the amino acid sequence of SEQ ID NO: 10 or 20; or an amino acid sequence which is at least 75%, 80%, 85%, 90%, 95%, 96%, 97%, 98%, 99%, or 100% identical to an amino acid of SEQ ID NO: 10 or SEQ ID NO: 20.

5. The VHH of claim 2, wherein
(i) plasmin is human plasmin;
(ii) the VHH binds to human plasmin with an affinity (KD) of 50 nM or less;
(iii) the VHH exhibits thrombolytic activity;
(iv) the VHH exhibits fibrinolysis-activating activity in plasma or whole blood;
(v) the VHH accelerates plasmin generation in vitro;
(vi) the VHH increases the conversion rate of a plasmin peptide substrate by plasmin in a dose-dependent manner;
(vii) the VHH increase tPA-induced plasmin generation in vitro; and/or
(viii) the VHH increases fibrin degradation rate in vitro.

6. A compound comprising at least one VHH of claim 2.

7. The compound of claim 6, wherein
(i) the compound further comprises one or more moieties selected from a chemical moiety providing for an extended half-life in vivo, a PEG moiety, an immunoglobulin which is not capable of specific binding to a human antigen, Transferrin, human Albumin, a homoamino acid polymer (HAP), a proline-alanine-serine (PAS) polymer, hydroxyethyl starch (HES), an elastin-like peptide (ELP), an Fc moiety, a peptide tag, an aptamer, a targeting moiety and an antibody or antibody fragment, and/or
(ii) one or more moieties are covalently or non-covalently bound to the at least one VHH, and/or
(iii) the compound is bispecific or multispecific, optionally wherein the compound further comprises at least one moiety specifically binding to a blood clot antigen.

8. A pharmaceutical composition comprising the VHH of claim 1.

9. A nucleic acid encoding the VHH of claim 2, or a vector comprising the nucleic acid.

10. A host cell comprising the nucleic acid or the vector of claim 9.

11. A method of producing the VHH of claim 2, comprising culturing a host cell comprising a nucleic acid the VHH or a vector comprising the nucleic acid under conditions suitable for the expression of the VHH.

12. A medical device comprising the VHH of claim 2, optionally wherein:
(i) the VHH is covalently or non-covalently coated onto the medical device, and/or
(ii) the medical device is an implantable medical device, and/or
(iii) the medical device is selected from a stent, a catheter or balloon catheter.

13. A method of treating a disease or condition characterized by a thrombotic or embolic state in a subject in need thereof comprising administering to the subject the VHH of claim 1.

14. The method of claim 13, wherein
(i) the disease or condition characterized by a thrombotic or embolic state is selected from embolism, lung embolism, stroke, infarction, brain infarction, thrombotic stroke, a microthrombotic disorder, venous occlusive disease, ischemia, thrombosis in dialysis patients, acute myocardial infarction, deep vein thrombosis, acute ischemic stroke, acute peripheral arterial occlusion, occlusion of indwelling catheters, intracardiac thrombus formation and microthrombotic ischemia; and/or
(ii) the subject is treated in an emergency medicine context, or wherein the disease or condition is an acute or chronic disease or condition; and/or
(iii) the VHH is administered parenterally,
and/or the VHH is formulated for parenteral administration.

15. The method of claim 13, wherein the VHH is used in combination with a second thrombolytic agent, optionally wherein:
(i) the second thrombolytic agent is selected from a tPA protein, an uPA protein, streptokinase, alteplase, reteplase, tenecteplase, urokinase, prourokinase, and anistreplase (APSAC), and/or
(ii) the second thrombolytic agent is administered to a subject at a lower dose in the combination as compared to the administration of the second thrombolytic agent alone.

16. A medical device comprising the compound of claim 6, optionally wherein:
(i) the compound is covalently or non-covalently coated onto the medical device, and/or
(ii) the medical device is an implantable medical device, and/or
(iii) the medical device is selected from a stent, a catheter or balloon catheter.

17. A medical device comprising the pharmaceutical composition of claim 8, optionally wherein:
(i) the pharmaceutical composition is covalently or non-covalently coated onto the medical device, and/or
(ii) the medical device is an implantable medical device, and/or
(iii) the medical device is selected from a stent, a catheter or balloon catheter.

18. A method of treating a disease or condition characterized by a thrombotic or embolic state in a subject in need thereof comprising administering to the subject the compound of claim 6.

19. A method of treating a disease or condition characterized by a thrombotic or embolic state in a subject in need thereof comprising administering to the subject the pharmaceutical composition of claim 8.

20. A method of treating a disease or condition characterized by a thrombotic or embolic state in a subject in need thereof comprising administering to the subject the medical device of claim 12.
